(19) United States
(12) Patent Application Publication
(10) Pub. No.: US 2023/0092578 A1
(43) Pub. Date: Mar. 23, 2023

(54) METHODS FOR PREPARING BISPHOSPHOCINS

(71) Applicant: Lakewood Amedex, Inc., University Park, FL (US)

(72) Inventor: Kelvin Cooper, Sarasota, FL (US)

(21) Appl. No.: 17/871,494

(22) Filed: Jul. 22, 2022

Related U.S. Application Data
(60) Provisional application No. 63/224,594, filed on Jul. 22, 2021.
(60) Provisional application No. 63/300,354, filed on Jan. 18, 2022.

(51) Int. Cl. C07H 19/10

(52) U.S. Cl. CPC C07H 19/10

(57) ABSTRACT

Methods for synthesizing Bisphosphocins use chemical modification of dialcoholic compounds avoiding the use of tetrazole and tertiary butyl hydroperoxide.

### CROSS-REFERENCE TO RELATED APPLICATIONS

This application claims priority to U.S. Provisional Patent Application No. 63/224,594, filed on Jul. 22, 2021, and U.S. Provisional Patent Application No. 63/300,354, filed on Jan. 18, 2022, the contents of which are incorporated herein by reference in their entireties.

### FIELD

The present disclosure provides methods for the preparation of Bisphosphocins®, including (2R,3S,5R)-2-((butoxy(hydroxy)phosphoryloxy)methyl)-5-(5-methyl-2,4-dioxo-3,4-di-hydroprimidin-1(2H)-yl)-tetrahydrofuran-3-yl)butyl phosphate, disodium salt (Nu-3) and (2R,3S,5R)-5-(4-amino-2-oxopyridin-1(2H)-yl)-3-(butoxy(hydroxy)phosphor-yloxy)tetrahydrofuran-2-yl)methyl butyl phosphate, disodium salt (Nu-8), avoiding the use of tetrazole and tertiary butyl hydroperoxide.

### BACKGROUND

Bisphosphocin compounds, including (2R,3S,5R)-2-((butoxy(hydroxy)phosphoryloxy)-methyl)-5-(5-methyl-2,4-dioxo-3,4-dihydroprimidin-1(2H)-yl)-tetrahydrofuran-3-yl)butyl phosphate, disodium salt (Nu-3, CAS #2254635-40-8) and (2R,3S,5R)-5-(4-amino-2-oxopyridin-1(2H)-yl)-3-(butoxy(hydroxy)phosphoryloxy)tetrahydrofuran-2-yl)methyl butyl phosphate, disodium salt (Nu-8, CAS #2222459-35-8), have therapeutic activity. U.S. Pat. No. 7,868,162 discloses Bisphosphocin compounds.

The existing process for the manufacture of Nu-8 is outlined in FIG. 1. While the route is capable of being scaled for the manufacture of Nu-3 and Nu-8 it presents some shortcomings. For example, the phosphatidylation reaction uses a six-fold excess of tetrazole as the activation agent, which would be potentially hazardous at commercial scale, and the conversion of the phosphite esters to the phosphonate esters uses an excess of tertiary butyl hydroperoxide, which would also be potentially hazardous at commercial scale. Nu-3 is manufactured using a similar process, but with thymidine as the nucleoside starting material instead of the bis(carbonyloxytertiary-butyl)(Boc)-protected cytidine used for Nu-8.

As a result, a need remains for an approach to the preparation of the Bisphosphocins that is efficient, inexpensive, occurs in good yield, and is safe to run at commercial scale.

### SUMMARY

The present disclosure provides a method for synthesizing a Bisphosphocin of Formula 1

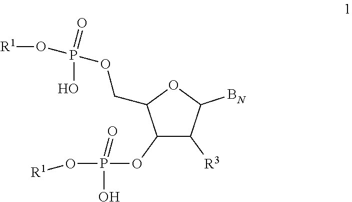

### or a Bisphosphocin of Formula 2

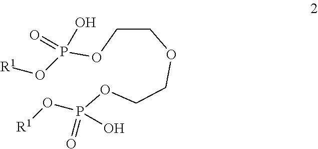

that includes: contacting tris(trifluoroethyl)phosphate 3

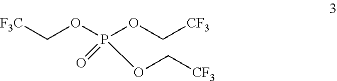 [SMILES: O=P(OCC(F)(F)F)(OCC(F)(F)F)OCC(F)(F)F]

with an alkyl alcohol R¹—OH under conditions sufficient to form a first mixed phosphate ester 4

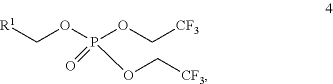

thereby producing the first mixed phosphate ester 4; contacting the first mixed phosphate ester 4 with a lithium alkoxide LiOR² or an allyl alcohol HOR² under conditions sufficient to form a second mixed phosphate ester 5

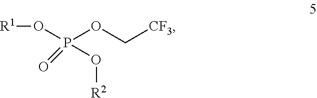

thereby producing the second mixed phosphate ester 5; contacting the second mixed phosphate ester 5 with a dialcohol of Formula 6

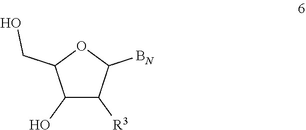

### or of Formula 7

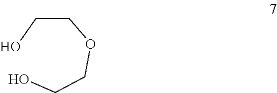

under conditions sufficient to form a protected Bisphosphocin of Formula 8

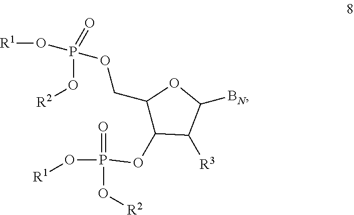

### or of Formula 9

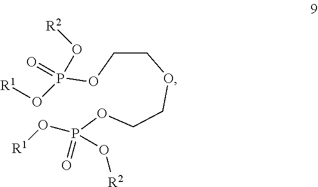

respectively, thereby producing the protected Bisphosphocin of Formula 8 or of Formula 9, respectively; and deprotecting the protected Bisphosphocin of Formula 8 or of Formula 9 under conditions sufficient to form the Bisphosphocin of Formula 1 or of Formula 2, respectively, thereby producing the Bisphosphocin of Formula 1 or of Formula 2, respectively; wherein: each R¹ is independently (CH₂)ₙCH₃ or (CH₂)ₙOH; each n is independently 2, 3, 4, 5, 6, 7, or 8; each R² is independently (CH₃)₃C—, CF₃CH₂—, PhCH₂—, CH₂═CHCH₂—, (CH3)₂CH—, CCl₃CH₂—, (CH3)₃SiCH₂CH₂—, 4-methoxy benzyl, C₆H₅SCH₂CH₂—, CH₃SO₂CH₂CH₂—, CH₃SCH₂CH₂CH₂CH₂—, and CF₃C(═O)N(CH₃)CH₂CH₂CH₂CH₂—; each R³ is independently hydrogen or methoxy; and B_{N} is a nitrogenous base.

In embodiments, the nitrogenous base comprises a purine, a pyrimidine, or a derivative thereof.

In embodiments, the nitrogenous base is selected from the group consisting of adenine, cytosine, guanine, thymine, and uracil.

In embodiments the Bisphosphocin of Formula 1 or the Bisphosphocin of Formula 2 is selected from the group consisting of a compound of Formula 10

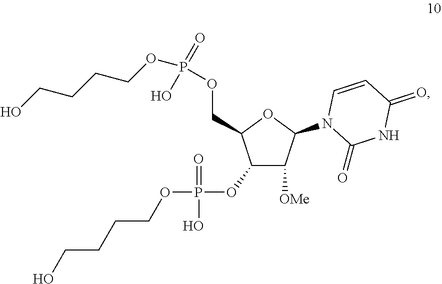 [SMILES: CO[C@@H]1[C@H](OP(=O)(O)OCCCCO)[C@@H](COP(=O)(O)OCCCCO)O[C@H]1n1ccc(=O)[nH]c1=O]

a compound of Formula 11

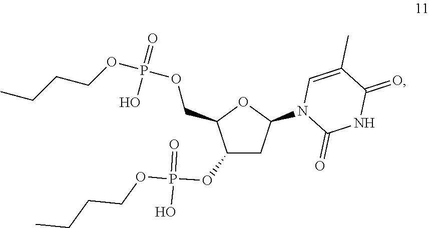 [SMILES: CCCCOP(=O)(O)OC[C@H]1O[C@@H](n2cc(C)c(=O)[nH]c2=O)C[C@@H]1OP(=O)(O)OCCCC]

a compound of Formula 12

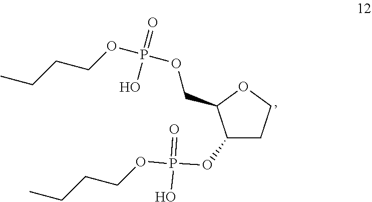 [SMILES: CCCCOP(=O)(O)OC[C@H]1OCC[C@@H]1OP(=O)(O)OCCCC]

a compound of Formula 13

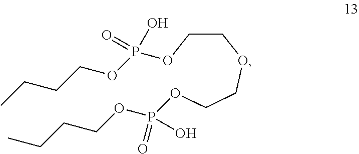 [SMILES: CCCCOP(=O)(O)OCCOCCOP(=O)(O)OCCCC]

and a compound of Formula 14

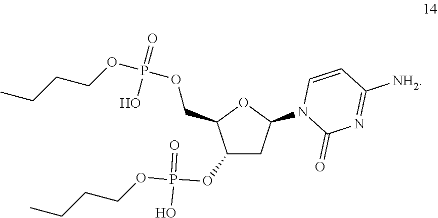 [SMILES: CCCCOP(=O)(O)OC[C@H]1O[C@@H](n2ccc(N)nc2=O)C[C@@H]1OP(=O)(O)OCCCC]

In embodiments, contacting tris(trifluoroethyl)phosphate 3 with the alkyl alcohol R¹—OH comprises: dissolving the tris(trifluoroethyl) phosphate 3 in a solvent to form a first solution; adding a non-nucleophilic base to the first solution; adding the alkyl alcohol R¹—OH to the first solution; and maintaining a temperature of the first solution from about −50° C. to about 50° C.

In embodiments, contacting the first mixed phosphate ester 4 with the lithium alkoxide LiOR² or the allyl alcohol HOR² comprises: dissolving the first mixed phosphate ester 4 in a solvent to form a second solution; adding a non-nucleophilic base to the second solution; adding the lithium alkoxide LiOR² or the allyl alcohol HOR² to the second solution; and maintaining a temperature of the second solution from about −50° C. to about 50° C.

In embodiments, contacting the second mixed phosphate ester 5 with a dialcohol of Formula 6 or of Formula 7 comprises: dissolving the dialcohol of Formula 6 or of Formula 7 in a solvent to form a third solution; adding an acid or a base to the third solution; adding the second mixed phosphate ester 5 to the third solution; and maintaining a temperature of the third solution from about −50° C. to about 50° C.

In embodiments, deprotecting the protected Bisphosphocin of Formula 8 or of Formula 9 comprises: dissolving the protected Bisphosphocin of Formula 8 or of Formula 9 in a solvent to form a fourth solution; adding an deprotection agent to the fourth solution; and maintaining a temperature of the fourth solution from about 40° C. to about 140° C.

In embodiments, the dialcohol of Formula 6 is a dialcohol of Formula 17

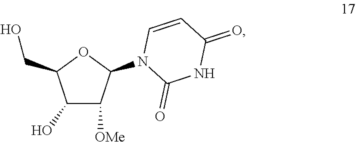 [SMILES: CO[C@@H]1[C@H](O)[C@@H](CO)O[C@H]1n1ccc(=O)[nH]c1=O]

### and the Bisphosphocin of Formula 1 is a Bisphosphocin of Formula 10

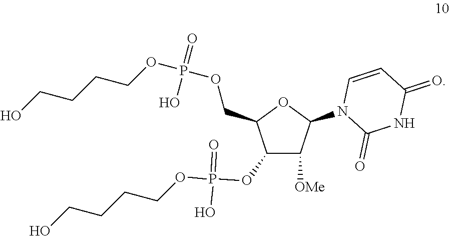 [SMILES: CO[C@@H]1[C@H](OP(=O)(O)OCCCCO)[C@@H](COP(=O)(O)OCCCCO)O[C@H]1n1ccc(=O)[nH]c1=O]

In embodiments, the dialcohol of Formula 6 is a dialcohol of Formula 18

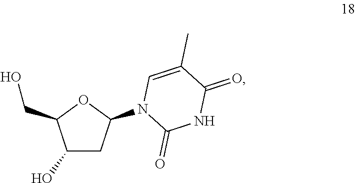 [SMILES: Cc1cn([C@H]2C[C@H](O)[C@@H](CO)O2)c(=O)[nH]c1=O]

### and the Bisphosphocin of Formula 1 is a Bisphosphocin of Formula 11

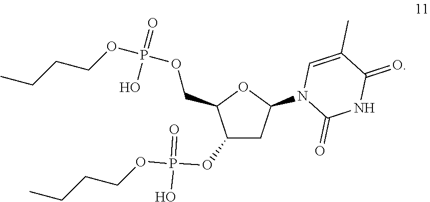 [SMILES: CCCCOP(=O)(O)OC[C@H]1O[C@@H](n2cc(C)c(=O)[nH]c2=O)C[C@@H]1OP(=O)(O)OCCCC]

In embodiments, the dialcohol of Formula 6 is a dialcohol of Formula 19

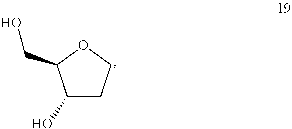

### and the Bisphosphocin of Formula 1 is a Bisphosphocin of Formula 12

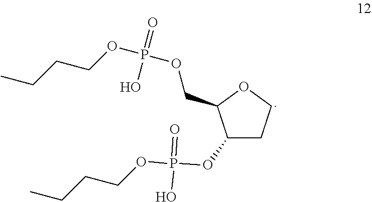 [SMILES: CCCCOP(=O)(O)OC[C@H]1OCC[C@@H]1OP(=O)(O)OCCCC]

In embodiments, the dialcohol is a dialcohol of Formula 7

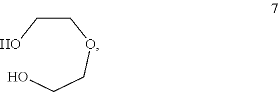

### and the Bisphosphocin of Formula 2 is a Bisphosphocin of Formula 13

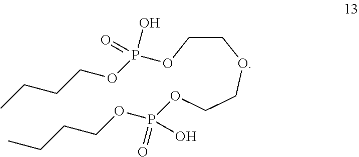 [SMILES: CCCCOP(=O)(O)OCCOCCOP(=O)(O)OCCCC]

In embodiments, the dialcohol of Formula 6 is a dialcohol of Formula 22

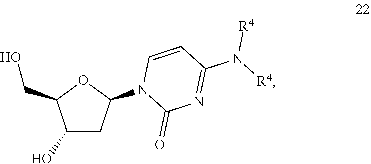

### and the Bisphosphocin of Formula 1 is a Bisphosphocin of Formula 14

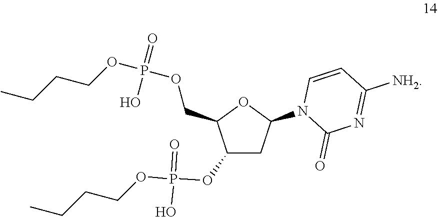

wherein each R⁴ is independently hydrogen, benzyloxycarbonyl, trichloroethoxycarbonyl, t-butoxycarbonyl, benzoyl, acetyl, and 9-fluorenylmethoxycarbonyl.

In embodiments, a method for synthesizing a Bisphosphocin of Formula 1

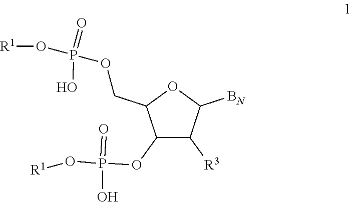

### or a Bisphosphocin of Formula 2

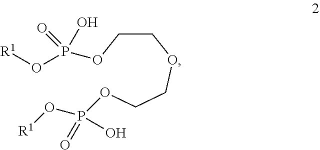

includes: contacting a dialcohol of Formula 6

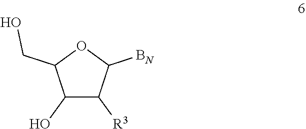

### or of Formula 7

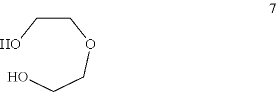

with phosphorus oxychloride in the presence of an alcohol of formula HO(CH₂)ₙCH₃ or HO(CH₂)ₙOH, under conditions sufficient to form the Bisphosphocin of Formula 1 or a Bisphosphocin of Formula 2, respectively; wherein each R¹ is independently (CH₂)ₙCH₃ or (CH₂)ₙOH; each n is independently 2, 3, 4, 5, 6, 7, or 8; each R³ is independently hydrogen or methoxy; and B_{N} is a nitrogenous base.

In embodiments, the nitrogenous base comprises a purine or a pyrimidine.

In embodiments, the nitrogenous base is selected from the group consisting of adenine, cytosine, guanine, thymine, and uracil.

In embodiments, the Bisphosphocin of Formula 1 or the Bisphosphocin of Formula 2 is selected from the group consisting of a compound of Formula 10

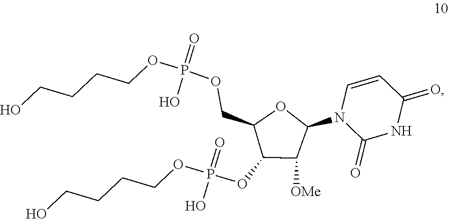 [SMILES: CO[C@@H]1[C@H](OP(=O)(O)OCCCCO)[C@@H](COP(=O)(O)OCCCCO)O[C@H]1n1ccc(=O)[nH]c1=O]

a compound of Formula 11

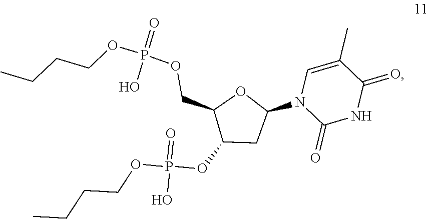 [SMILES: CCCCOP(=O)(O)OC[C@H]1O[C@@H](n2cc(C)c(=O)[nH]c2=O)C[C@@H]1OP(=O)(O)OCCCC]

a compound of Formula 12

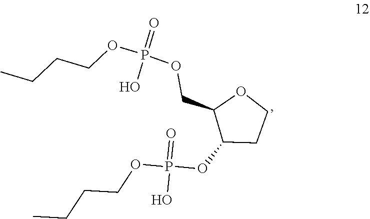 [SMILES: CCCCOP(=O)(O)OC[C@H]1OCC[C@@H]1OP(=O)(O)OCCCC]

a compound of Formula 13

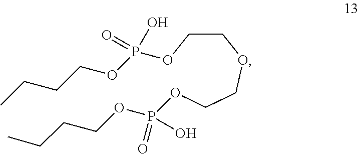 [SMILES: CCCCOP(=O)(O)OCCOCCOP(=O)(O)OCCCC]

a compound of Formula 14

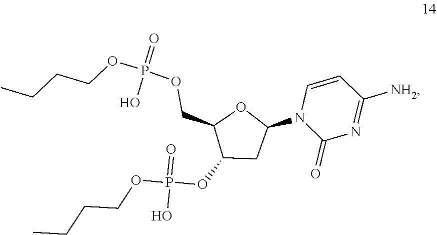 [SMILES: CCCCOP(=O)(O)OC[C@H]1O[C@@H](n2ccc(N)nc2=O)C[C@@H]1OP(=O)(O)OCCCC]

and a compound of Formula 23:

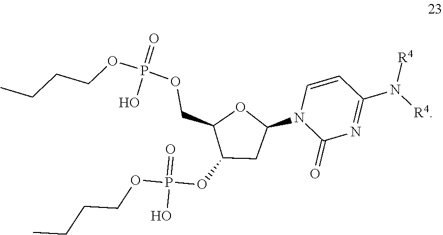

In embodiments, contacting the dialcohol of Formula 6 or of Formula 7 with the phosphorus oxychloride comprises: dissolving the dialcohol of Formula 6 or of Formula 7 in a mixture of trialkyl phosphate and phosphorus oxychloride; stirring the mixture at a temperature from about −20° C. to about 20° C. for a period of time from about 10 minutes to about 3 hours; adding the alcohol of formula HO(CH₂)ₙCH₃ or HO(CH₂)ₙOH to the mixture; and stirring the mixture at a temperature from about −20° C. to about 20° C. for a period of time from 1 hour to 10 hours.

In embodiments, the alcohol of formula HO(CH₂)ₙCH₃ is butanol.

In embodiments, the alcohol of formula HO(CH₂)ₙOH is 1,4-butanediol.

In embodiments, the dialcohol of Formula 6 is a dialcohol of Formula 17

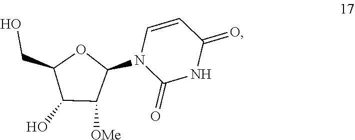 [SMILES: CO[C@@H]1[C@H](O)[C@@H](CO)O[C@H]1n1ccc(=O)[nH]c1=O]

### and the Bisphosphocin of Formula 1 is a Bisphosphocin of Formula 10

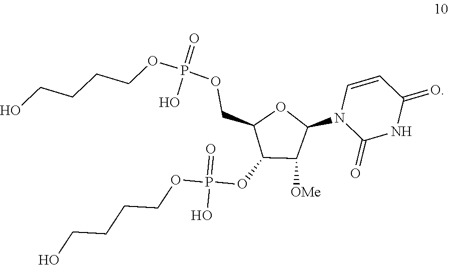 [SMILES: CO[C@@H]1[C@H](OP(=O)(O)OCCCCO)[C@@H](COP(=O)(O)OCCCCO)O[C@H]1n1ccc(=O)[nH]c1=O]

In embodiments, the dialcohol of Formula 6 is a dialcohol of Formula 18

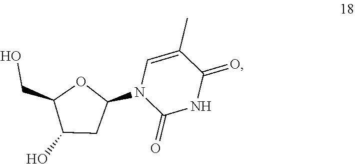 [SMILES: Cc1cn([C@H]2C[C@H](O)[C@@H](CO)O2)c(=O)[nH]c1=O]

### and the Bisphosphocin of Formula 1 is a Bisphosphocin of Formula 11

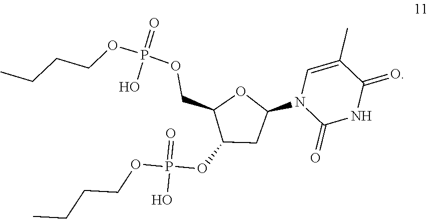 [SMILES: CCCCOP(=O)(O)OC[C@H]1O[C@@H](n2cc(C)c(=O)[nH]c2=O)C[C@@H]1OP(=O)(O)OCCCC]

In embodiments, the dialcohol of Formula 6 is a dialcohol of Formula 19

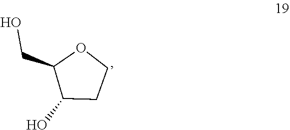

### and the Bisphosphocin of Formula 1 is a Bisphosphocin of Formula 12

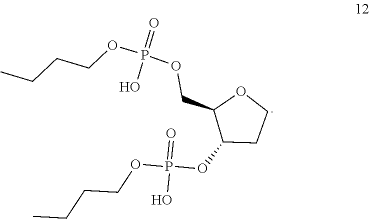 [SMILES: CCCCOP(=O)(O)OC[C@H]1OCC[C@@H]1OP(=O)(O)OCCCC]

In embodiments, the dialcohol is a dialcohol of Formula 7

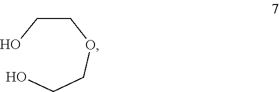

### and the Bisphosphocin of Formula 2 is a Bisphosphocin of Formula 13

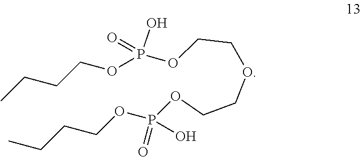 [SMILES: CCCCOP(=O)(O)OCCOCCOP(=O)(O)OCCCC]

In embodiments, the dialcohol of Formula 6 is a dialcohol of Formula 22

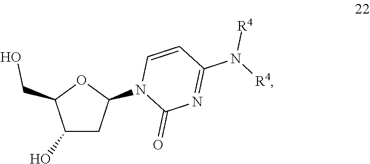

### and the Bisphosphocin of Formula 1 is a Bisphosphocin of Formula 23

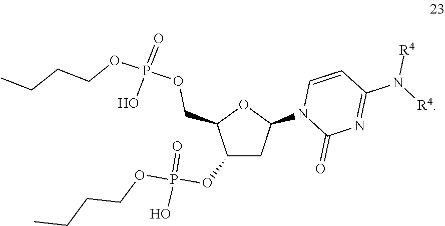

wherein each R⁴ is independently hydrogen, benzyloxycarbonyl, trichloroethoxycarbonyl, t-butoxycarbonyl, benzoyl, acetyl, and 9-fluorenylmethoxycarbonyl.

In embodiments, the method further comprises deprotecting the Bisphosphocin of Formula

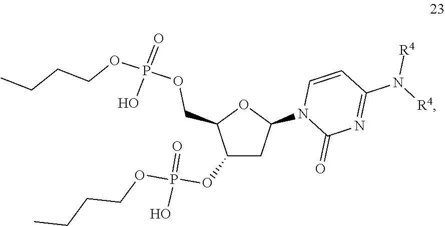

thereby producing a Bisphosphocin of Formula 14:

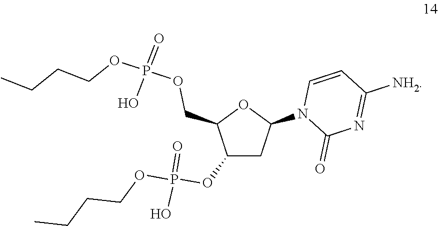 [SMILES: CCCCOP(=O)(O)OC[C@H]1O[C@@H](n2ccc(N)nc2=O)C[C@@H]1OP(=O)(O)OCCCC]

Additional aspects and embodiments will be apparent from the Detailed Description and from the claims.

### BRIEF DESCRIPTION OF THE DRAWINGS

The teachings of some embodiments of the present disclosure will be better understood by reference to the description taken in conjunction with the accompanying drawings, wherein:

FIG. 1 shows a traditional reaction scheme for the production of Nu-8;

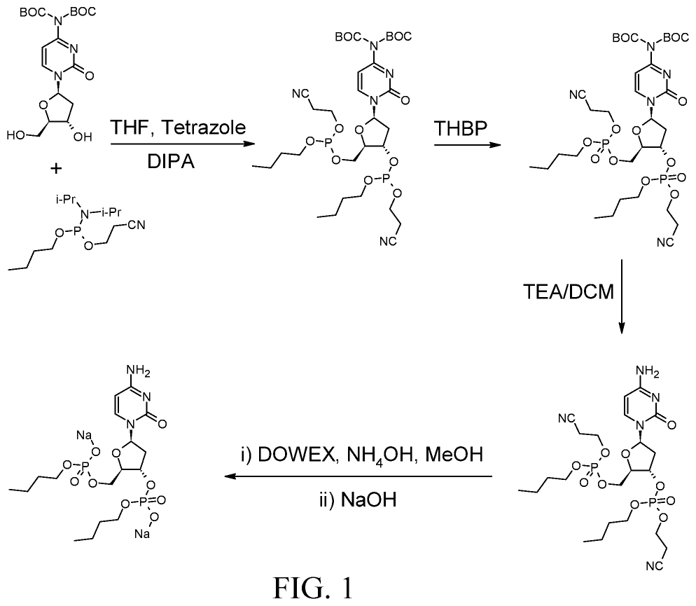

FIG. 2 shows the ¹H NMR spectrum of compound 38 according to embodiments;

FIG. 3 shows the ¹³C NMR spectrum of compound 38 according to embodiments;

FIG. 4 shows the ³¹P NMR spectrum of compound 38 according to embodiments;

FIG. 5 shows the mass spectrum of compound 38 according to embodiments;

FIG. 6 shows the ¹H NMR spectrum of compound 39 according to embodiments;

FIG. 7 shows the ¹³C NMR spectrum of compound 39 according to embodiments;

FIG. 8 shows the ³¹P NMR spectrum of compound 39 according to embodiments;

FIG. 9 shows the mass spectrum of compound 39 according to embodiments;

FIG. 10 shows the ¹H NMR spectrum of compound 36 according to embodiments;

FIG. 11 shows the ¹H NMR spectrum of compound 37 according to embodiments;

FIG. 12 shows the ¹H NMR spectrum of compound 43 according to embodiments;

FIG. 13 shows the ¹H NMR spectrum of compound 42 according to embodiments;

FIG. 14 shows the ¹H NMR spectrum of compound 39 according to embodiments;

FIG. 15 shows the ¹³C NMR spectrum of compound 39 according to embodiments;

FIG. 16 shows the ³¹P NMR spectrum of compound 39 according to embodiments;

FIG. 17 shows the mass spectrum of compound 39 according to embodiments;

FIG. 18 shows the ¹H NMR spectrum of compound 14 according to embodiments;

FIG. 19 shows the ¹³C NMR spectrum of compound 14 according to embodiments;

FIG. 20 shows the ³¹P NMR spectrum of compound 14 according to embodiments;

FIG. 21 shows the mass spectrum of compound 14 according to embodiments;

FIG. 22 shows the ¹H NMR spectrum of compound 45 according to embodiments;

FIG. 23 shows the ¹H NMR spectrum of compound 46 according to embodiments;

FIG. 24 shows the ¹H NMR spectrum of compound 47 according to embodiments;

FIG. 25 shows the ¹H NMR spectrum of compound 14 according to embodiments;

FIG. 26 shows the ¹³C NMR spectrum of compound 14 according to embodiments;

FIG. 27 shows the ³¹P NMR spectrum of compound 14 according to embodiments;

FIG. 28 shows the mass spectrum of compound 14 according to embodiments;

FIG. 29 shows the ¹H NMR spectrum of compound 50 according to embodiments;

FIG. 30 shows the ¹H NMR spectrum of compound 48 according to embodiments;

FIG. 31 shows the ¹H NMR spectrum of compound 53 according to embodiments; and

FIG. 32 shows the ¹H NMR spectrum of compound 55 according to embodiments.

### DETAILED DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

The present disclosure provides methods that avoid the use of tetrazole and tertiary butyl hydroperoxide for synthesizing a Bisphosphocin of Formula 1 or a Bisphosphocin of Formula 2:

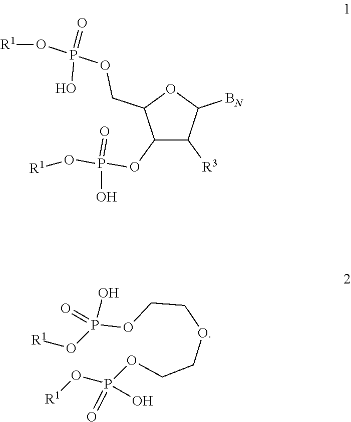

In embodiments, the Bisphosphocin of Formula 1 or the Bisphosphocin of Formula 2 is selected from a compound of Formula 10, a compound of Formula 11, a compound of Formula 12, a compound of Formula 13, and a compound of Formula 14:

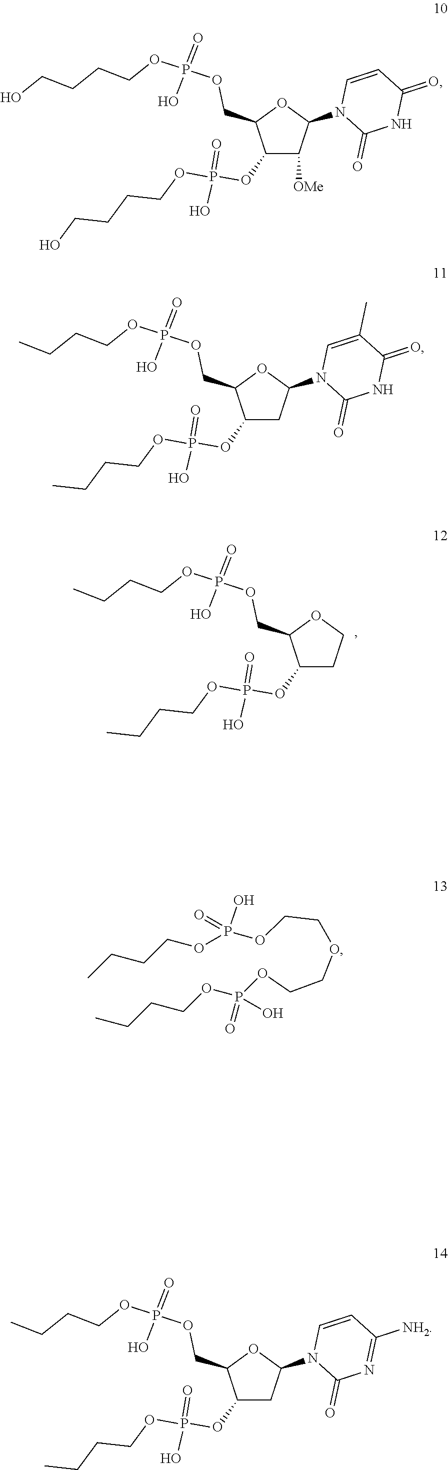 [SMILES: CO[C@@H]1[C@H](OP(=O)(O)OCCCCO)[C@@H](COP(=O)(O)OCCCCO)O[C@H]1n1ccc(=O)[nH]c1=O ; CCCCOP(=O)(O)OC[C@H]1O[C@@H](n2cc(C)c(=O)[nH]c2=O)C[C@@H]1OP(=O)(O)OCCCC ; CCCCOP(=O)(O)OC[C@H]1OCC[C@@H]1OP(=O)(O)OCCCC ; CCCCOP(=O)(O)OCCOCCOP(=O)(O)OCCCC ; CCCCOP(=O)(O)OC[C@H]1O[C@@H](n2ccc(N)nc2=O)C[C@@H]1OP(=O)(O)OCCCC]

The chemical name of the compound of Formula 10 is (4-hydroxybutyl)-phosphate-5′-uridine-2′-methoxy-3′-phosphate-(4-hydroxybutyl). The molecular formula of the compound of Formula 10 is C₁₈H₃₀N₂O₁₄P₂²⁻ when the phosphate groups are in their deprotonated form. The molecular weight of the compound of Formula 10 is 560.38 Da when the phosphate groups are in their deprotonated form. The compound of Formula 10 is also referred to herein as Nu-2, which such terms are used interchangeably herein. In some embodiments, a compound of Formula 10 includes a ribose, two phosphate groups, two hydroxybutyl groups, and a uracil.

The chemical name of the compound of Formula 11 is butyl-phosphate-5′-thymidine-3′-phosphate-butyl. The molecular formula of the compound of Formula 11 is C₁₈H₃₀N₂O₁₁P₂²⁻ when the phosphate groups are in their deprotonated form. The molecular weight of the compound of Formula 11 is 512.39 Da when the phosphate groups are in their deprotonated form. The compound of Formula 11 is also referred to herein as Nu-3, which such terms are used interchangeably herein. In some embodiments, a compound of Formula 11 includes a ribose, two phosphate groups, two butyl groups, and a thymine.

The chemical name of the compound of Formula 12 is butyl-phosphate-5′-ribose-3′-phosphate-butyl. The molecular formula of the compound of Formula 12 is C₁₃H₂₆O₉P₂²⁻ when the phosphate groups are in their deprotonated form. The molecular weight of the compound of Formula 12 is 388.29 Da when the phosphate groups are in their deprotonated form. The compound of Formula 12 is also referred to herein as Nu-4, which such terms are used interchangeably herein. In some embodiments, a compound of Formula 12 includes a ribose, two phosphate groups, and two butyl groups.

The chemical name of the compound of Formula 13 is P,P′-(oxydi-2,1-ethanediyl)bis(P-butyl phosphate) molecular formula of the compound of Formula 13 is C₁₂H₂₆O₉P₂²⁻ when the phosphate groups are in their deprotonated form. The molecular weight of the compound of Formula 13 is 376.28 Da when the phosphate groups are in their deprotonated form. The compound of Formula 13 is also referred to herein as Nu-5, which such terms are used interchangeably herein. In some embodiments, a compound of Formula 13 includes two phosphate groups and two butyl groups.

The chemical name of the compound of Formula 14 is ((2R,3S,5R)-5-(4-amino-2-oxopyrimidin-1(2H)-yl)-3-((butoxyoxidophosphor-yl)oxy)tetrahydrofuran-2-yl)methyl butyl phosphate. The molecular formula of the compound of Formula 14 is C₁₇H₂₉N₃Na₂O₁₀P₂²⁻ when the phosphate groups are in their deprotonated form. The molecular weight of the compound of Formula 14 is 497.37 Da when the phosphate groups are in their deprotonated form. The compound of Formula 14 is also referred to herein as Nu-8, which such terms are used interchangeably herein. In some embodiments, a compound of the present disclosure includes a ribose, two phosphate groups, two butyl groups, and a cytosine.

It is understood by those skilled in the art that some compounds may exhibit tautomerism. In such cases, the formulae provided herein expressly depict only one of the possible tautomeric forms. It is therefore to be understood that the compound of Formula (I) intends to represent any tautomeric form of the depicted compound and is not to be limited merely to the specific tautomeric form depicted by the drawing of the compound.

### Process A

In an embodiment, the method includes contacting tris(trifluoroethyl)phosphate 3 with an alkyl alcohol R¹—OH under conditions sufficient to form a first mixed phosphate ester 4, thereby producing the first mixed phosphate ester 4:

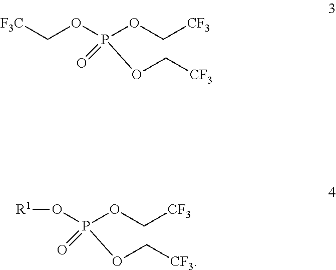

The method also includes contacting the first mixed phosphate ester 4 with a lithium alkoxide LiOR² or an allyl alcohol HOR² under conditions sufficient to form a second mixed phosphate ester 5, thereby producing the second mixed phosphate ester 5:

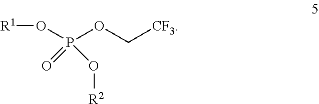

Additionally, the method includes contacting the second mixed phosphate ester 5 with a dialcohol of Formula 6 or of Formula 7 under conditions sufficient to form a protected Bisphosphocin of Formula 8 or of Formula 9, respectively, thereby producing the protected Bisphosphocin of Formula 8 or of Formula 9, respectively:

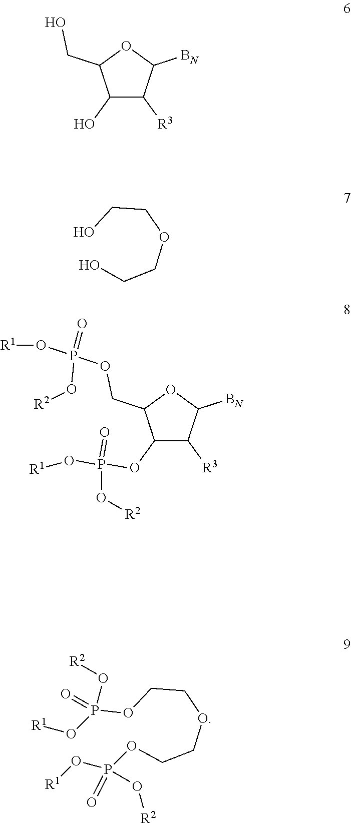

The method also includes deprotecting the protected Bisphosphocin of Formula 8 or of Formula 9 under conditions sufficient to form the Bisphosphocin of Formula 1 or of Formula 2, respectively, thereby producing the Bisphosphocin of Formula 1 or of Formula 2, respectively. In each of Formula 1 through Formula 9, each R¹ is independently (CH₂)ₙCH₃ or (CH₂)ₙOH; each n is independently 2, 3, 4, 5, 6, 7, or 8; each R² is independently (CH₃)₃C—, CF₃CH₂—, PhCH₂—, CH₂═CHCH₂—, (CH3)₂CH—, CCl₃CH₂—, (CH3)₃SiCH₂CH₂—, 4-methoxy benzyl, C₆H₅SCH₂CH₂—, CH₃SO₂CH₂CH₂—, CH₃SCH₂CH₂CH₂CH₂—, and CF₃C(═O)N(CH₃)CH₂CH₂CH₂CH₂—; each R³ is independently hydrogen or methoxy; and B_{N} is a nitrogenous base. The method will now be described in additional detail.

In embodiments, the nitrogenous base, B_{N}, may be a purine or a pyrimidine. A pyrimidine is a monocyclic heteroaromatic organic compound with a nitrogen atom at the 1-position and the 3-position. A purine is a heterocyclic aromatic organic compound containing a fused ring system of pyrimidine and imidazole. Both pyrimidine and purine may bear substituents on the ring system, and may include other derivative forms. Unsubstituted pyrimidine is shown as Formula 24, and unsubstituted purine is shown as Formula 25. In embodiments, the nitrogenous base may be one or more of adenine 26, cytosine 27, guanine 28, thymine 29, and uracil 30.

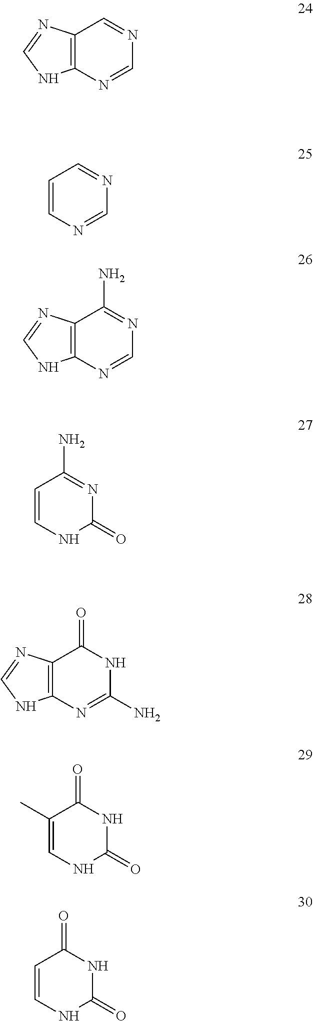

As noted above, the method includes contacting tris(trifluoroethyl)phosphate 3 with an alkyl alcohol R¹—OH under conditions sufficient to form a first mixed phosphate ester 4, thereby producing the first mixed phosphate ester 4. In embodiments, this contacting may include dissolving the tris(trifluoroethyl)phosphate 3 in a solvent to form a first solution; adding a non-nucleophilic base to the first solution; adding the alkyl alcohol R¹—OH to the first solution; and maintaining a temperature of the first solution from about −50° C. to about 50° C.

In embodiments, the solvent used to form the first solution may be, for example, an aromatic solvent such as benzene, toluene, or xylene (ortho, meta, para, or any mixture thereof); tetrahydrofuran (THF); dioxane; dimethylformamide (DMF); a hydrocarbon solvent such as any combination of isomers of heptane, hexane, or octane, including pure straight-chain isomers; a halocarbon solvent such as dichloromethane or chloroform; or a combination of two or more thereof.

In embodiments, the non-nucleophilic base may be, for example, an amine or a nitrogen heterocycle. The category of amines and nitrogen heterocycles includes, but is not limited to, N,N-diisopropylethylamine (DIPEA), 1,8-diazabicycloundec-7-ene (DBU), 1,5-diazabicyclo[4.3.0]-non-5-ene (DBN), and 2,6-di-tert-butylpyridine.

In embodiments, the alkyl alcohol R¹—OH may be one or more of HO(CH₂)ₙCH₃ or HO(CH₂)ₙOH; each n is independently 2, 3, 4, 5, 6, 7, or 8. For example, and without limitation, the alkyl alcohol may be ethan-1-ol; propan-1-ol; butan-1-ol; pentan-1-ol; hexan-1-ol; heptan-1-ol; octan-1-ol; nonan-1-ol; 1,2-diethanol; 1,3-dipropanol; 1,4-dibutanol; 1,5-dipentanol; 1,6-dihexanol; 1,7-diheptanol; 1,8-dioctanol; or any combination of two or more of these.

In embodiments, the temperature of the first solution may be maintained from about −50° C. to about 50° C. For example, the temperature may be maintained from about −50° C. to about 45° C., from about −50° C. to about 40° C., from about −50° C. to about 35° C., from about −50° C. to about 30° C., from about −50° C. to about 25° C., from about −50° C. to about 20° C., from about −50° C. to about 15° C., from about −50° C. to about 10° C., from about −50° C. to about 5° C., from about −50° C. to about 0° C., from about −50° C. to about −5° C., from about −50° C. to about −10° C., from about −50° C. to about −15° C., from about −50° C. to about −20° C., from about −50° C. to about −25° C., from about −50° C. to about −30° C., from about −50° C. to about −35° C., from about −50° C. to about −40° C., from about −50° C. to about −45° C., from about −45° C. to about 50° C., from about −40° C. to about 50° C., from about −35° C. to about 50° C., from about −30° C. to about 50° C., from about −25° C. to about 50° C., from about −20° C. to about 50° C., from about −15° C. to about 50° C., from about −10° C. to about 50° C., from about −5° C. to about 50° C., from about 0° C. to about 50° C., from about 5° C. to about 50° C., from about 10° C. to about 50° C., from about 15° C. to about 50° C., from about 15° C. to about 30° C., from about 20° C. to about 50° C., from about 25° C. to about 50° C., from about 30° C. to about 50° C., from about 35° C. to about 50° C., from about 40° C. to about 50° C., or even from about 45° C. to about 50° C.

As noted above, the method includes contacting the first mixed phosphate ester 4 with a lithium alkoxide LiOR² or an allyl alcohol HOR² under conditions sufficient to form a second mixed phosphate ester 5, thereby producing the second mixed phosphate ester 5. In embodiments, this contacting may include dissolving the first mixed phosphate ester 4 in a solvent to form a second solution; adding a non-nucleophilic base to the second solution; adding the lithium alkoxide LiOR² or the allyl alcohol HOR² to the second solution; and maintaining a temperature of the second solution from about −50° C. to about 50° C.

In embodiments the lithium alkoxide may comprise one or more of (CH₃)₃COLi, CF₃CH₂OLi, PhCH₂OLi, and CH₂═CHCH₂OLi. In embodiments, the allyl alcohol may comprise CH₂═CHCH₂OH.

In embodiments, the solvent used to form the second solution may be, for example, an aromatic solvent such as benzene, toluene, or xylene (ortho, meta, para, or any mixture thereof); tetrahydrofuran (THF); dioxane; dimethylformamide (DMF); a hydrocarbon solvent such as any combination of isomers of heptane, hexane, or octane, including pure straight-chain isomers; a halocarbon solvent such as dichloromethane or chloroform; or a combination of two or more thereof.

In embodiments, the temperature of the second solution may be maintained from about −50° C. to about 50° C. For example, the temperature may be maintained from about −50° C. to about 45° C., from about −50° C. to about 40° C., from about −50° C. to about 35° C., from about −50° C. to about 30° C., from about −50° C. to about 25° C., from about −50° C. to about 20° C., from about −50° C. to about 15° C., from about −50° C. to about 10° C., from about −50° C. to about 5° C., from about −50° C. to about 0° C., from about −50° C. to about −5° C., from about −50° C. to about −10° C., from about −50° C. to about −15° C., from about −50° C. to about −20° C., from about −50° C. to about −25° C., from about −50° C. to about −30° C., from about −50° C. to about −35° C., from about −50° C. to about −40° C., from about −50° C. to about −45° C., from about −45° C. to about 50° C., from about −40° C. to about 50° C., from about −35° C. to about 50° C., from about −30° C. to about 50° C., from about −25° C. to about 50° C., from about −20° C. to about 50° C., from about −15° C. to about 50° C., from about −10° C. to about 50° C., from about −5° C. to about 50° C., from about 0° C. to about 50° C., from about 5° C. to about 50° C., from about 10° C. to about 50° C., from about 15° C. to about 50° C., from about 15° C. to about 30° C., from about 20° C. to about 50° C., from about 25° C. to about 50° C., from about 30° C. to about 50° C., from about 35° C. to about 50° C., from about 40° C. to about 50° C., or even from about 45° C. to about 50° C.

As noted above, the method includes contacting the second mixed phosphate ester 5 with a dialcohol of Formula 6 or of Formula 7 under conditions sufficient to form a protected Bisphosphocin of Formula 8 or of Formula 9, respectively, thereby producing the protected Bisphosphocin of Formula 8 or of Formula 9, respectively. In embodiments, this contacting may include dissolving the dialcohol of Formula 6 or of Formula 7 in a solvent to form a third solution; adding an acid or a base to the third solution; adding the second mixed phosphate ester 5 to the third solution; and maintaining a temperature of the third solution from about −50° C. to about 50° C.

In embodiments, the solvent used to form the third solution may be, for example, an aromatic solvent such as benzene, toluene, or xylene (ortho, meta, para, or any mixture thereof); tetrahydrofuran (THF); dioxane; dimethylformamide (DMF); a hydrocarbon solvent such as any combination of isomers of heptane, hexane, or octane, including pure straight-chain isomers; a halocarbon solvent such as dichloromethane or chloroform; or a combination of two or more thereof.

In embodiments using a base, the base may be selected from strong silazide bases, such as sodium hexamethyldisilazide, strong amide bases such as lithium diispropylamide or lithium tetramethylpiperidide, or strong metal hydrides such as sodium hydride, or alkyllithiums such as n-butyl lithium or tertiary butyl lithium.

In embodiments, the temperature of the third solution may be maintained from about −50° C. to about 50° C. For example, the temperature may be maintained from about −50° C. to about 45° C., from about −50° C. to about 40° C., from about −50° C. to about 35° C., from about −50° C. to about 30° C., from about −50° C. to about 25° C., from about −50° C. to about 20° C., from about −50° C. to about 15° C., from about −50° C. to about 10° C., from about −50° C. to about 5° C., from about −50° C. to about 0° C., from about −50° C. to about −5° C., from about −50° C. to about −10° C., from about −50° C. to about −15° C., from about −50° C. to about −20° C., from about −50° C. to about −25° C., from about −50° C. to about −30° C., from about −50° C. to about −35° C., from about −50° C. to about −40° C., from about −50° C. to about −45° C., from about −45° C. to about 50° C., from about −40° C. to about 50° C., from about −35° C. to about 50° C., from about −30° C. to about 50° C., from about −25° C. to about 50° C., from about −20° C. to about 50° C., from about −15° C. to about 50° C., from about −10° C. to about 50° C., from about −5° C. to about 50° C., from about 0° C. to about 50° C., from about 5° C. to about 50° C., from about 10° C. to about 50° C., from about 15° C. to about 50° C., from about 15° C. to about 30° C., from about 20° C. to about 50° C., from about 25° C. to about 50° C., from about 30° C. to about 50° C., from about 35° C. to about 50° C., from about 40° C. to about 50° C., or even from about 45° C. to about 50° C.

As noted above, the method includes deprotecting the protected Bisphosphocin of Formula 8 or of Formula 9 under conditions sufficient to form the Bisphosphocin of Formula 1 or of Formula 2, respectively, thereby producing the Bisphosphocin of Formula 1 or of Formula 2, respectively. In embodiments, this deprotecting includes dissolving the protected Bisphosphocin of Formula 8 or of Formula 9 in a solvent to form a fourth solution; adding a deprotection agent to the fourth solution; and maintaining a temperature of the fourth solution from about 40° C. to about 140° C.

In embodiments, the solvent used to form the fourth solution may be, for example, an aromatic solvent such as benzene, toluene, or xylene (ortho, meta, para, or any mixture thereof); acetone; tetrahydrofuran (THF); dioxane; dimethylformamide (DMF); a hydrocarbon solvent such as any combination of isomers of heptane, hexane, or octane, including pure straight-chain isomers; a halocarbon solvent such as dichloromethane or chloroform; or a combination of two or more thereof.

In embodiments, the deprotection agent may comprise H₂, sodium iodide, tetrakis(triphenylphospine)palladium, trifluoro acetic acid, dilute hydrochloric acid, sodium hydroxide, sodium methoxide, sodium ethoxide, zinc-copper couple, tertiary-butyl ammonium fluoride, trimethylsilyl bromide, tris(triphenylphosphine)rhodium chloride, ammonium hydroxide, sodium periodate-sodium hydroxide, HF-pyridine, and PtO₂.

In embodiments, the temperature of the fourth solution may be maintained from about 40° C. to about 140° C. For example, the temperature may be maintained from about 40° C. to about 135° C., from about 40° C. to about 130° C., from about 40° C. to about 125° C., from about 40° C. to about 120° C., from about 40° C. to about 115° C., from about 40° C. to about 110° C., from about 40° C. to about 105° C., from about 40° C. to about 100° C., from about 40° C. to about 95° C., from about 40° C. to about 90° C., from about 40° C. to about 85° C., from about 40° C. to about 80° C., from about 40° C. to about 75° C., from about 40° C. to about 70° C., from about 40° C. to about 65° C., from about 40° C. to about 60° C., from about 40° C. to about 55° C., from about 40° C. to about 50° C., from about 40° C. to about 45° C., from about 45° C. to about 140° C., from about 50° C. to about 140° C., from about 55° C. to about 140° C., from about 60° C. to about 140° C., from about 65° C. to about 140° C., from about 70° C. to about 140° C., from about 75° C. to about 140° C., from about 80° C. to about 140° C., from about 85° C. to about 140° C., from about 90° C. to about 140° C., from about 95° C. to about 140° C., from about 100° C. to about 140° C., from about 105° C. to about 140° C., from about 110° C. to about 140° C., from about 115° C. to about 140° C., from about 120° C. to about 140° C., from about 125° C. to about 140° C., from about 130° C. to about 140° C., or even from about 135° C. to about 140° C.

In embodiments, the dialcohol of Formula 6 is a dialcohol of Formula 17 and the resulting Bisphosphocin of Formula 1 is a Bisphosphocin of Formula 10. In embodiments, the dialcohol of Formula 6 is a dialcohol of Formula 18 and the resulting Bisphosphocin of Formula 1 is a Bisphosphocin of Formula 11. In embodiments, the dialcohol of Formula 6 is a dialcohol of Formula 19 and the resulting Bisphosphocin of Formula 1 is a Bisphosphocin of Formula 12. In embodiments, the dialcohol is a dialcohol of Formula 7 and the resulting Bisphosphocin of Formula 2 is a Bisphosphocin of Formula 13. In embodiments, the dialcohol of Formula 6 is a dialcohol of Formula 22 and the resulting Bisphosphocin of Formula 1 is a Bisphosphocin of Formula 14 (upon deprotection of the cytidine amino group, which may be protected throughout the method such that a compound of Formula 23 may be converted to a compound of Formula 14).

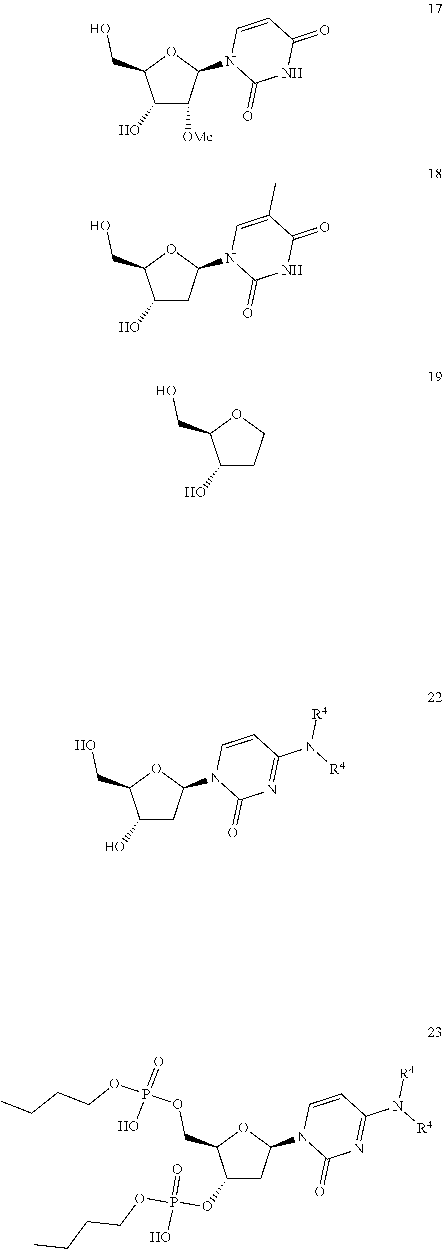

The method will now be further elucidated via a detailed discussion of the synthesis of the compound of Formula 11, as shown in Scheme 1.

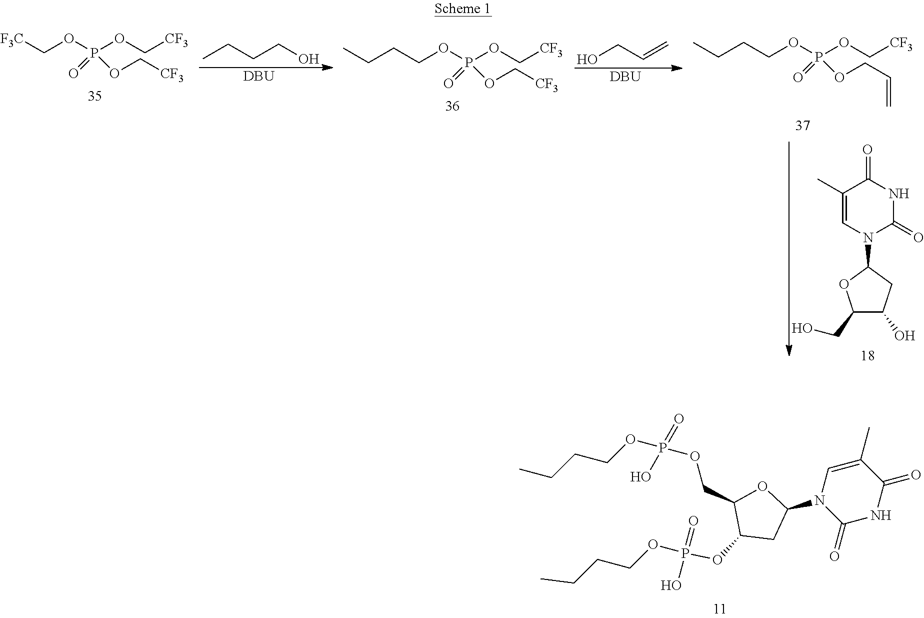

The use of phosphorus coupling agents that employ 5-valent phosphorus avoids the sensitive nature of 3-valent phosphorus reagents to moisture and oxygen. Tris(2,2,2-trifluoroethyl)phosphate has been used as a versatile reagent for preparing mixed unsymmetrical phosphate triesters. Scheme 1 shows a pathway for generating the particular unsymmetrical esters needed to prepare the reagents for the synthesis of Bisphosphocins 11 and 14.

The unsymmetrical phosphate ester 37 can be prepared two ways. Firstly, as shown in Scheme 1, commercially available tris-trifluoroethyl phosphonate 35 may be converted to the butyl analog 36 with either 1,8-diazabicyclo[5.4.0]undec-7-ene and n-butanol or lithium butoxide in toluene at −45° C., followed by treatment of the intermediate with a further, different alkoxide, such as allyl alcohol, also at −45° C. to produce 37. Alternatively, introduction of the butyl group and allyl group could be reversed, such that commercially available tris-trifluoroethyl phosphonate 35 may be converted to the allyl analog by reacting 35 with the required alkoxide (e.g., allyl alcohol) and DBU or alkali metal alkoxide. The thus prepared compound may then be reacted with either DBU/butanol or lithium butoxide in toluene or THF at −45° C.

The unsymmetrical phosphate ester 37 can then be reacted with the nucleoside 18, which bears a dialcohol moiety, and a suitable strong base, such as sodium hexamethyldisilazide, at low temperature in a suitable solvent, such as THE or THF/DMF to produce the compound of Formula 11. The compound of Formula 14 may be formed similarly using nucleoside 22 instead of nucleoside 18, followed by deprotection of the cytidine amino group:

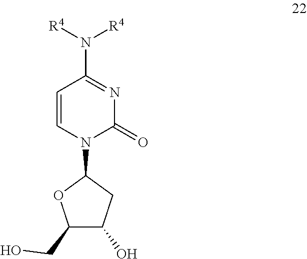

where each R⁴ is an amine protecting group. Exemplary amine protecting groups include, but are not limited to, benzyloxycarbonyl, trichloroethoxycarbonyl, tertiary-butoxycarbonyl, benzoyl, acetyl, and 9-fluorenylmethoxycarbonyl.

Deprotection of the cytidine amino group may be accomplished in accordance with techniques and reagents known to one of skill in the art. For instance, in embodiments, one R⁴ is H and one R⁴ is benzylcarbonyl, which may be removed using a sodium hydroxide solution. In embodiments, one R⁴ is H and one R⁴ is tricholorethoxycarbonyl, which may be removed using zinc and a dilute hydrochloric acid solution. In embodiments, both R⁴ are tertiary-butoxycarbonyl, which may be removed using a dilute hydrochloric acid solution. In embodiments, one R⁴ is H and one R⁴ is benzoyl, which may be removed with sodium hydroxide solution. In embodiments, one R⁴ is H and one R⁴ is acetyl, which may be removed with sodium hydroxide solution. In embodiments, one R⁴ is H and one R⁴ is 9-fluorenylmethoxycarbonyl, which may be removed with ammonium hydroxide solution. Of course, other amino protecting groups and deprotection protocols are envisioned.

### Process B

In embodiments, a method for synthesizing a Bisphosphocin of Formula 1 or a Bisphosphocin of Formula 2 includes contacting a dialcohol of Formula 6 or of Formula 7 with phosphorus oxychloride in the presence of an alcohol of formula HO(CH₂)ₙCH₃ or HO(CH₂)ₙOH, under conditions sufficient to form the Bisphosphocin of Formula 1 or a Bisphosphocin of Formula 2, respectively.

As with Process A, in embodiments, the nitrogenous base, B_{N}, may be a purine or a pyrimidine. Unsubstituted pyrimidine is shown as Formula 24, and unsubstituted purine is shown as Formula 25. In embodiments, the nitrogenous base may be one or more of adenine 26, cytosine 27, guanine 28, thymine 29, and uracil 30.

Also as in Process A, in embodiments, the Bisphosphocin of Formula 1 of the Bisphosphocin of Formula 2 is selected from a compound of Formula 10, a compound of Formula 11, a compound of Formula 12, a compound of Formula 13, and a compound of Formula 14.

In embodiments, contacting the dialcohol of Formula 6 or of Formula 7 with the phosphorus oxychloride may include dissolving the dialcohol of Formula 6 or of Formula 7 in a mixture of trialkyl phosphate and phosphorus oxychloride; stirring the mixture at a temperature from about −20° C. to about 20° C. for a period of time from about 10 minutes to about 3 hours; adding the alcohol of formula HO(CH₂)ₙCH₃ or HO(CH₂)ₙOH to the mixture; and stirring the mixture at a temperature from about −20° C. to about 20° C. for a period of time from about 1 hour to about 10 hours.

As noted above, in certain embodiments, the mixture with or without the alcohol may be stirred at a temperature from about −20° C. to about 20° C. That is, the temperature may be from about −20° C. to about 19° C., from about −20° C. to about 18° C., from about −20° C. to about 17° C., from about −20° C. to about 16° C., from about −20° C. to about 15° C., from about −20° C. to about 14° C., from about −20° C. to about 13° C., from about −20° C. to about 12° C., from about −20° C. to about 11° C., from about −20° C. to about 10° C., from about −20° C. to about 9° C., from about −20° C. to about 8° C., from about −20° C. to about 7° C., from about −20° C. to about 6° C., from about −20° C. to about 5° C., from about −20° C. to about 4° C., from about −20° C. to about 3° C., from about −20° C. to about 2° C., from about −20° C. to about 1° C., from about −20° C. to about 0° C., from about −20° C. to about −1° C., from about −20° C. to about −2° C., from about −20° C. to about −3° C., from about −20° C. to about −4° C., from about −20° C. to about −5° C., from about −20° C. to about −6° C., from about −20° C. to about −7° C., from about −20° C. to about −8° C., from about −20° C. to about −9° C., from about −20° C. to about −10° C., from about −20° C. to about −11° C., from about −20° C. to about −12° C., from about −20° C. to about −13° C., from about −20° C. to about −14° C., from about −20° C. to about −15° C., from about −20° C. to about −16° C., from about −20° C. to about −17° C., from about −20° C. to about −18° C., from about −20° C. to about −19° C., from about −19° C. to about 20° C., from about −18° C. to about 20° C., from about −17° C. to about 20° C., from about −16° C. to about 20° C., from about −15° C. to about 20° C., from about −14° C. to about 20° C., from about −13° C. to about 20° C., from about −12° C. to about 20° C., from about −11° C. to about 20° C., from about −10° C. to about 20° C., from about −9° C. to about 20° C., from about −8° C. to about 20° C., from about −7° C. to about 20° C., from about −6° C. to about 20° C., from about −5° C. to about 20° C., from about −4° C. to about 20° C., from about −3° C. to about 20° C., from about −2° C. to about 20° C., from about −1° C. to about 20° C., from about 0° C. to about 20° C., from about 1° C. to about 20° C., from about 2° C. to about 20° C., from about 3° C. to about 20° C., from about 14° C. to about 20° C., from about 5° C. to about 20° C., from about 6° C. to about 20° C., from about 7° C. to about 20° C., from about 8° C. to about 20° C., from about 9° C. to about 20° C., from about 10° C. to about 20° C., from about 11° C. to about 20° C., from about 12° C. to about 20° C., from about 13° C. to about 20° C., from about 14° C. to about 20° C., from about 15° C. to about 20° C., from about 16° C. to about 20° C., from about 17° C. to about 20° C., from about 18° C. to about 20° C., or even from about 19° C. to about 20° C.

In embodiments, prior to adding the alcohol, the mixture may be stirred for from about 10 minutes to about three hours (180 minutes). That is, prior to adding the alcohol, the mixture may be stirred for from about 10 minutes to about 170 minutes, from about 10 minutes to about 160 minutes, from about 10 minutes to about 150 minutes, from about 10 minutes to about 140 minutes, from about 10 minutes to about 130 minutes, from about 10 minutes to about 120 minutes, from about 10 minutes to about 110 minutes, from about 10 minutes to about 100 minutes, from about 10 minutes to about 90 minutes, from about 10 minutes to about 80 minutes, from about 10 minutes to about 70 minutes, from about 10 minutes to about 60 minutes, from about 10 minutes to about 50 minutes, from about 10 minutes to about 40 minutes, from about 10 minutes to about 30 minutes, from about 10 minutes to about 20 minutes, from about 20 minutes to about 180 minutes, from about 30 minutes to about 180 minutes, from about 40 minutes to about 180 minutes, from about 50 minutes to about 180 minutes, from about 60 minutes to about 180 minutes, from about 70 minutes to about 180 minutes, from about 80 minutes to about 180 minutes, from about 90 minutes to about 180 minutes, from about 100 minutes to about 180 minutes, from about 110 minutes to about 180 minutes, from about 120 minutes to about 180 minutes, from about 130 minutes to about 180 minutes, from about 140 minutes to about 180 minutes, from about 150 minutes to about 180 minutes, from about 160 minutes to about 180 minutes, or even from about 170 minutes to about 180 minutes.

In embodiments, the alcohol may be of formula HO(CH₂)ₙCH₃ or HO(CH₂)ₙOH; each n is independently 2, 3, 4, 5, 6, 7, or 8. For example, the alcohol may be ethan-1-ol; propan-1-ol; butan-1-01; pentan-1-ol; hexan-1-ol; heptan-1-ol; octan-1-ol; nonan-1-ol; 1,2-diethanol; 1,3-dipropanol; 1,4-dibutanol; 1,5-dipentanol; 1,6-dihexanol; 1,7-diheptanol; 1,8-dioctanol; or any combination of two or more of these.

Upon adding the alcohol to the mixture, the mixture may be stirred at a temperature from about −20° C. to about 20° C. That is, after adding the alcohol, the temperature may be from about −20° C. to about 19° C., from about −20° C. to about 18° C., from about −20° C. to about 17° C., from about −20° C. to about 16° C., from about −20° C. to about 15° C., from about −20° C. to about 14° C., from about −20° C. to about 13° C., from about −20° C. to about 12° C., from about −20° C. to about 11° C., from about −20° C. to about 10° C., from about −20° C. to about 9° C., from about −20° C. to about 8° C., from about −20° C. to about 7° C., from about −20° C. to about 6° C., from about −20° C. to about 5° C., from about −20° C. to about 4° C., from about −20° C. to about 3° C., from about −20° C. to about 2° C., from about −20° C. to about 1° C., from about −20° C. to about 0° C., from about −20° C. to about −1° C., from about −20° C. to about −2° C., from about −20° C. to about −3° C., from about −20° C. to about −4° C., from about −20° C. to about −5° C., from about −20° C. to about −6° C., from about −20° C. to about −7° C., from about −20° C. to about −8° C., from about −20° C. to about −9° C., from about −20° C. to about −10° C., from about −20° C. to about −11° C., from about −20° C. to about −12° C., from about −20° C. to about −13° C., from about −20° C. to about −14° C., from about −20° C. to about −15° C., from about −20° C. to about −16° C., from about −20° C. to about −17° C., from about −20° C. to about −18° C., from about −20° C. to about −19° C., from about −19° C. to about 20° C., from about −18° C. to about 20° C., from about −17° C. to about 20° C., from about −16° C. to about 20° C., from about −15° C. to about 20° C., from about −14° C. to about 20° C., from about −13° C. to about 20° C., from about −12° C. to about 20° C., from about −11° C. to about 20° C., from about −10° C. to about 20° C., from about −9° C. to about 20° C., from about −8° C. to about 20° C., from about −7° C. to about 20° C., from about −6° C. to about 20° C., from about −5° C. to about 20° C., from about −4° C. to about 20° C., from about −3° C. to about 20° C., from about −2° C. to about 20° C., from about −1° C. to about 20° C., from about 0° C. to about 20° C., from about 1° C. to about 20° C., from about 2° C. to about 20° C., from about 3° C. to about 20° C., from about 14° C. to about 20° C., from about 5° C. to about 20° C., from about 6° C. to about 20° C., from about 7° C. to about 20° C., from about 8° C. to about 20° C., from about 9° C. to about 20° C., from about 10° C. to about 20° C., from about 11° C. to about 20° C., from about 12° C. to about 20° C., from about 13° C. to about 20° C., from about 14° C. to about 20° C., from about 15° C. to about 20° C., from about 16° C. to about 20° C., from about 17° C. to about 20° C., from about 18° C. to about 20° C., or even from about 19° C. to about 20° C.

In embodiments, this second stirring may be for an additional 1 hour to about 10 hours. That is, this second stirring may be for from about 1 hour to about 9.5 hours, from about 1 hour to about 9 hours, from about 1 hour to about 8.5 hours, from about 1 hour to about 8 hours, from about 1 hour to about 7.5 hours, from about 1 hour to about 7 hours, from about 1 hour to about 6.5 hours, from about 1 hour to about 6 hours, from about 1 hour to about 5.5 hours, from about 1 hour to about 5 hours, from about 1 hour to about 4.5 hours, from about 1 hour to about 4.5 hours, from about 1 hour to about 4 hours, from about 1 hour to about 3.5 hours, from about 1 hour to about 3 hours, from about 1 hour to about 2.5 hours, from about 1 hour to about 2 hours, from about 1 hour to about 1.5 hours, from about 1.5 hours to about 10 hours, from about 2 hours to about 10 hours, from about 2.5 hours to about 10 hours, from about 3 hours to about 10 hours, from about 3.5 hours to about 10 hours, from about 4 hours to about 10 hours, from about 4.5 hours to about 10 hours, from about 5 hours to about 10 hours, from about 5.5 hours to about 10 hours, from about 6 hours to about 10 hours, from about 6.5 hours to about 10 hours, from about 7 hours to about 10 hours, from about 7.5 hours to about 10 hours, from about 8 hours to about 10 hours, from about 8.5 hours to about 10 hours, from about 9 hours to about 10 hours, or even from about 9.5 hours to about 10 hours.

In embodiments, the dialcohol of Formula 6 is a dialcohol of Formula 17 and the resulting Bisphosphocin of Formula 1 is a Bisphosphocin of Formula 10. In embodiments, the dialcohol of Formula 6 is a dialcohol of Formula 18 and the resulting Bisphosphocin of Formula 1 is a Bisphosphocin of Formula 11. In embodiments, the dialcohol of Formula 6 is a dialcohol of Formula 19 and the resulting Bisphosphocin of Formula 1 is a Bisphosphocin of Formula 12. In embodiments, the dialcohol is a dialcohol of Formula 7 and the resulting Bisphosphocin of Formula 2 is a Bisphosphocin of Formula 13. In embodiments, the dialcohol of Formula 6 is a dialcohol of Formula 20 and the resulting Bisphosphocin of Formula 1 is a Bisphosphocin of Formula 14. In embodiments, the dialcohol of Formula 6 is a dialcohol of Formula 21 and the resulting Bisphosphocin of Formula 1 is a Bisphosphocin of Formula 15. In embodiments, the dialcohol of Formula 6 is a dialcohol of Formula 22 and the resulting Bisphosphocin of Formula 1 is a Bisphosphocin of Formula 14 (upon deprotection of the cytidine amino group, which may be protected throughout the method such that a compound of Formula 23 may be converted to a compound of Formula 14).

The method will now be further elucidated via a detailed discussion of the synthesis of the compound of Formula 14, as shown in Scheme 2.

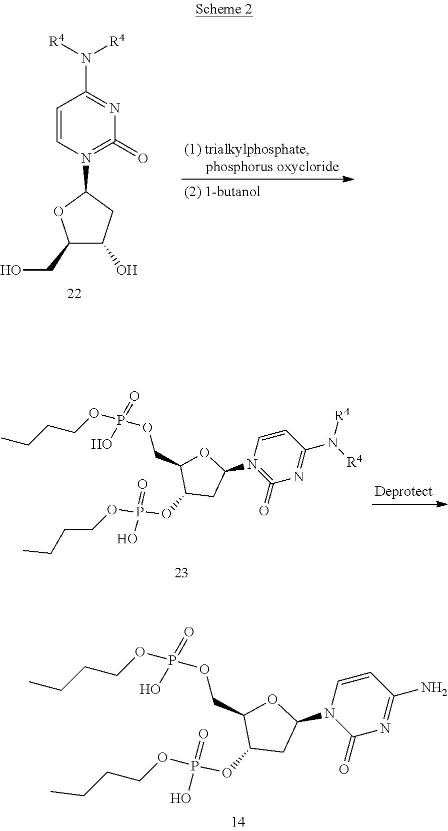

Reaction of 2′-deoxycytidine derivative 22 with a mixture of trialkyl phosphate and phosphorus oxychloride followed by the addition of n-butanol at low temperature affords the Bisphosphocin derivatives 23. As noted above, deprotection of the cytidine amino group gives the compound of Formula 14. For instance, in embodiments, one R⁴ is H and one R⁴ is benzylcarbonyl, which may be removed using a sodium hydroxide solution. In embodiments, one R⁴ is H and one R⁴ is tricholorethoxycarbonyl, which may be removed using zinc and a dilute hydrochloric acid solution. In embodiments, both R⁴ are tertiary-butoxycarbonyl, which may be removed using a dilute hydrochloric acid solution. Of course, other amino protecting groups and deprotection protocols are envisioned.

In addition to the aspects and embodiments described and provided elsewhere in the present disclosure, the following non-limiting list of embodiments are also contemplated.

1. A method for synthesizing a Bisphosphocin of Formula 1

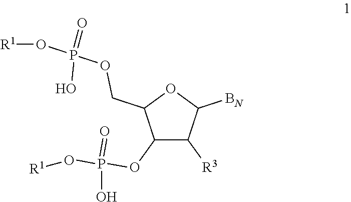

### or a Bisphosphocin of Formula 2

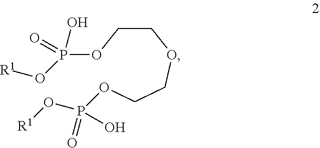

the method comprising:

contacting tris(trifluoroethyl)phosphate 3

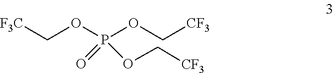 [SMILES: O=P(OCC(F)(F)F)(OCC(F)(F)F)OCC(F)(F)F]

with an alkyl alcohol R¹—OH under conditions sufficient to form a first mixed phosphate ester 4

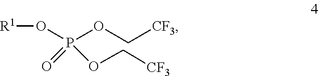

thereby producing the first mixed phosphate ester 4,

contacting the first mixed phosphate ester 4 with a lithium alkoxide LiOR² or an allyl alcohol HOR² under conditions sufficient to form a second mixed phosphate ester 5

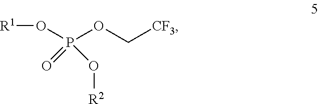

thereby producing the second mixed phosphate ester 5;

contacting the second mixed phosphate ester 5 with a dialcohol of Formula 6

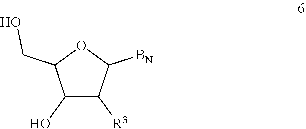

### or of Formula 7

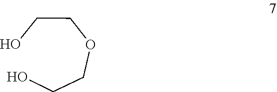

under conditions sufficient to form a protected Bisphosphocin of Formula 8

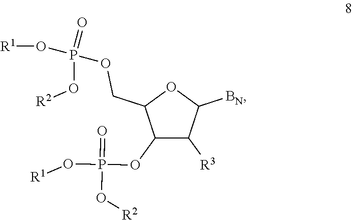

### or of Formula 9

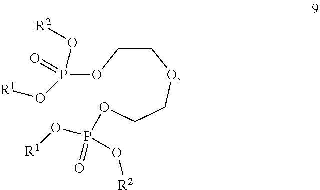

respectively, thereby producing the protected Bisphosphocin of Formula 8 or of Formula 9, respectively; and

deprotecting the protected Bisphosphocin of Formula 8 or of Formula 9 under conditions sufficient to form the Bisphosphocin of Formula 1 or of Formula 2, respectively, thereby producing the Bisphosphocin of Formula 1 or of Formula 2, respectively;

wherein:

each R¹ is independently (CH₂)ₙCH₃ or (CH₂)ₙOH;

each n is independently 2, 3, 4, 5, 6, 7, or 8;

each R² is independently (CH₃)₃C—, CF₃CH₂—, PhCH₂—, CH₂═CHCH₂—, (CH3)₂CH—, CCl₃CH₂—, (CH3)₃SiCH₂CH₂—, 4-methoxy benzyl, C₆H₅SCH₂CH₂—, CH₃SO₂CH₂CH₂—, CH₃SCH₂CH₂CH₂CH₂—, and CF₃C(═O)N(CH₃)CH₂CH₂CH₂CH₂—;

each R³ is independently hydrogen or methoxy; and

B_{N} is a nitrogenous base.

2. The method of clause 1, wherein the nitrogenous base comprises a purine, a pyrimidine, or a derivative thereof.

3. The method of clause 1 or clause 2, wherein the nitrogenous base is selected from the group consisting of adenine, cytosine, guanine, thymine, and uracil.

4. The method of any one of clauses 1-3, wherein the Bisphosphocin of Formula 1 or the Bisphosphocin of Formula 2 is selected from the group consisting of a compound of Formula 10

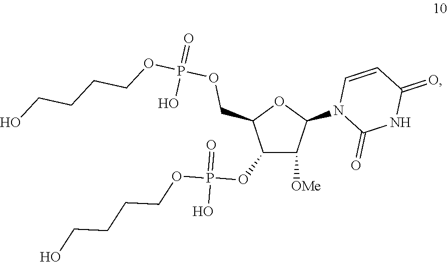 [SMILES: CO[C@@H]1[C@H](OP(=O)(O)OCCCCO)[C@@H](COP(=O)(O)OCCCCO)O[C@H]1n1ccc(=O)[nH]c1=O]

a compound of Formula 11

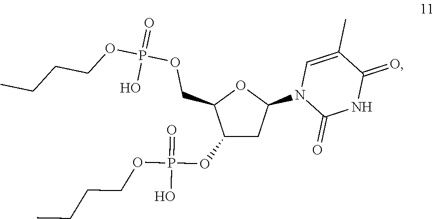 [SMILES: CCCCOP(=O)(O)OC[C@H]1O[C@@H](n2cc(C)c(=O)[nH]c2=O)C[C@@H]1OP(=O)(O)OCCCC]

a compound of Formula 12

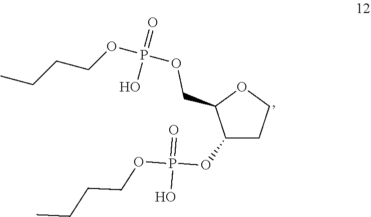 [SMILES: CCCCOP(=O)(O)OC[C@H]1OCC[C@@H]1OP(=O)(O)OCCCC]

a compound of Formula 13

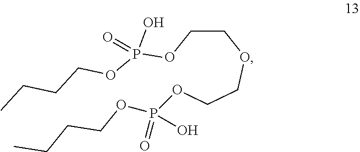 [SMILES: CCCCOP(=O)(O)OCCOCCOP(=O)(O)OCCCC]

and a compound of Formula 14

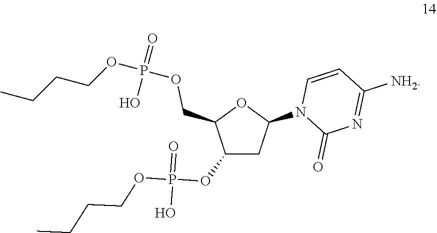 [SMILES: CCCCOP(=O)(O)OC[C@H]1O[C@@H](n2ccc(N)nc2=O)C[C@@H]1OP(=O)(O)OCCCC]

5. The method of any one of clauses 1-4, wherein contacting tris(trifluoroethyl)phosphate 3 with the alkyl alcohol R¹—OH comprises:

dissolving the tris(trifluoroethyl)phosphate 3 in a solvent to form a first solution;

adding a non-nucleophilic base to the first solution;

adding the alkyl alcohol R¹—OH to the first solution; and

maintaining a temperature of the first solution from about −50° C. to about 50° C.

6. The method of clause 5, wherein contacting the first mixed phosphate ester 4 with the lithium alkoxide LiOR² or the allyl alcohol HOR² comprises:

dissolving the first mixed phosphate ester 4 in a solvent to form a second solution;

adding a non-nucleophilic base to the second solution;

adding the lithium alkoxide LiOR² or the allyl alcohol HOR² to the second solution; and

maintaining a temperature of the second solution from about −50° C. to about 50° C.

7. The method of clause 6, wherein contacting the second mixed phosphate ester 5 with a dialcohol of Formula 6 or of Formula 7 comprises:

dissolving the dialcohol of Formula 6 or of Formula 7 in a solvent to form a third solution;

adding an acid or a base to the third solution;

adding the second mixed phosphate ester 5 to the third solution; and

maintaining a temperature of the third solution from about −50° C. to about 50° C.

8. The method of clause 7, wherein deprotecting the protected Bisphosphocin of Formula 8 or of Formula 9 comprises:

dissolving the protected Bisphosphocin of Formula 8 or of Formula 9 in a solvent to form a fourth solution;

adding an deprotection agent to the fourth solution; and

maintaining a temperature of the fourth solution from about 40° C. to about 140° C.

9. The method of any one of clauses 1-8, wherein the dialcohol of Formula 6 is a dialcohol of Formula 17

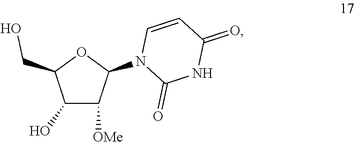 [SMILES: CO[C@@H]1[C@H](O)[C@@H](CO)O[C@H]1n1ccc(=O)[nH]c1=O]

and the Bisphosphocin of Formula 1 is a Bisphosphocin of Formula 10

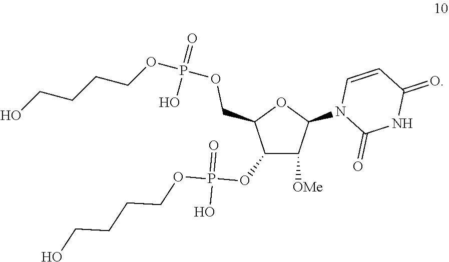 [SMILES: CO[C@@H]1[C@H](OP(=O)(O)OCCCCO)[C@@H](COP(=O)(O)OCCCCO)O[C@H]1n1ccc(=O)[nH]c1=O]

10. The method of any one of clauses 1-8, wherein the dialcohol of Formula 6 is a dialcohol of Formula 18

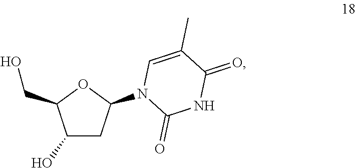 [SMILES: Cc1cn([C@H]2C[C@H](O)[C@@H](CO)O2)c(=O)[nH]c1=O]

and the Bisphosphocin of Formula 1 is a Bisphosphocin of Formula 11

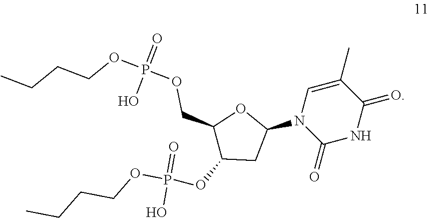 [SMILES: CCCCOP(=O)(O)OC[C@H]1O[C@@H](n2cc(C)c(=O)[nH]c2=O)C[C@@H]1OP(=O)(O)OCCCC]

11. The method of any one of clauses 1-8, wherein the dialcohol of Formula 6 is a dialcohol of Formula 19

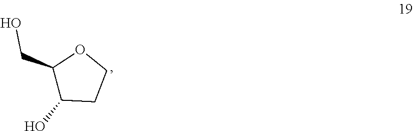

and the Bisphosphocin of Formula 1 is a Bisphosphocin of Formula 12

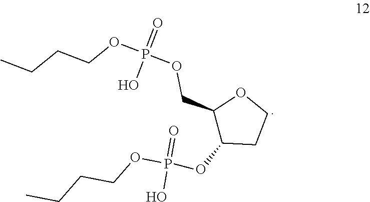 [SMILES: CCCCOP(=O)(O)OC[C@H]1OCC[C@@H]1OP(=O)(O)OCCCC]

12. The method of any one of clauses 1-8, wherein the dialcohol is a dialcohol of Formula 7

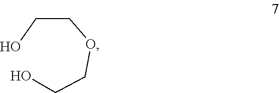

and the Bisphosphocin of Formula 2 is a Bisphosphocin of Formula 13

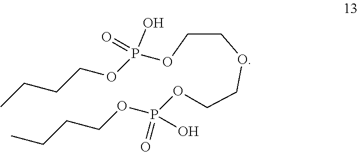 [SMILES: CCCCOP(=O)(O)OCCOCCOP(=O)(O)OCCCC]

13. The method of any one of clauses 1-8, wherein the dialcohol of Formula 6 is a dialcohol of Formula 22

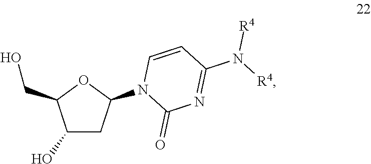

and the Bisphosphocin of Formula 1 is a Bisphosphocin of Formula 14

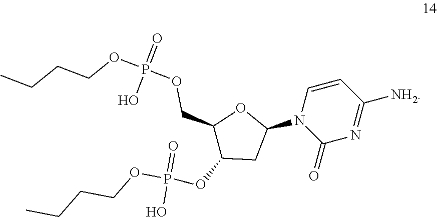

wherein each R⁴ is independently hydrogen, benzyloxycarbonyl, trichloroethoxycarbonyl, t-butoxycarbonyl, benzoyl, acetyl, and 9-fluorenylmethoxycarbonyl.

14. A method for synthesizing a Bisphosphocin of Formula 1

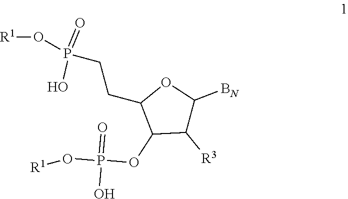

### or a Bisphosphocin of Formula 2

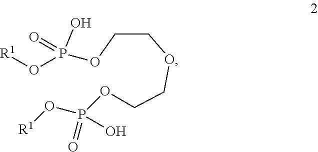

the method comprising:

contacting a dialcohol of Formula 6

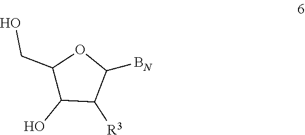

### or of Formula 7

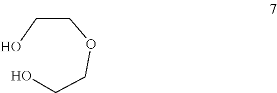

with phosphorus oxychloride in the presence of an alcohol of formula HO(CH₂)ₙCH₃ or HO(CH₂)ₙOH, under conditions sufficient to form the Bisphosphocin of Formula 1 or a Bisphosphocin of Formula 2, respectively;

wherein

each R¹ is independently (CH₂)CH₃ or (CH₂)ₙOH;

each n is independently 2, 3, 4, 5, 6, 7, or 8;

each R³ is independently hydrogen or methoxy; and

B_{N} is a nitrogenous base.

15. The method of clause 14, wherein the nitrogenous base comprises a purine or a pyrimidine.

16. The method of clause 14 or clause 15, wherein the nitrogenous base is selected from the group consisting of adenine, cytosine, guanine, thymine, and uracil.

17. The method of clause 14 or clause 15, wherein the Bisphosphocin of Formula 1 or the Bisphosphocin of Formula 2 is selected from the group consisting of a compound of Formula 10

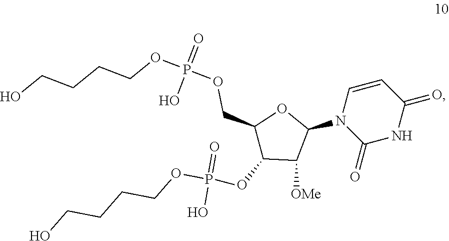 [SMILES: CO[C@@H]1[C@H](OP(=O)(O)OCCCCO)[C@@H](COP(=O)(O)OCCCCO)O[C@H]1n1ccc(=O)[nH]c1=O]

a compound of Formula 11

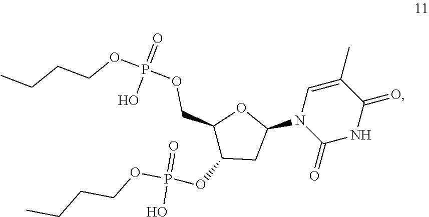 [SMILES: CCCCOP(=O)(O)OC[C@H]1O[C@@H](n2cc(C)c(=O)[nH]c2=O)C[C@@H]1OP(=O)(O)OCCCC]

a compound of Formula 12

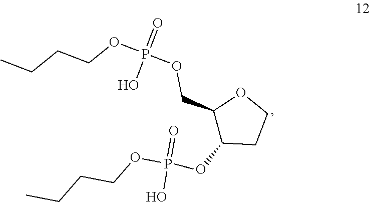 [SMILES: CCCCOP(=O)(O)OC[C@H]1OCC[C@@H]1OP(=O)(O)OCCCC]

a compound of Formula 13

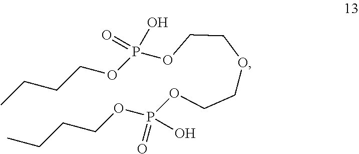 [SMILES: CCCCOP(=O)(O)OCCOCCOP(=O)(O)OCCCC]

a compound of Formula 14

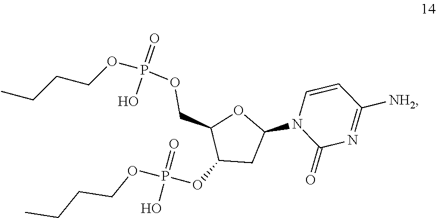 [SMILES: CCCCOP(=O)(O)OC[C@H]1O[C@@H](n2ccc(N)nc2=O)C[C@@H]1OP(=O)(O)OCCCC]

and a compound of Formula 23:

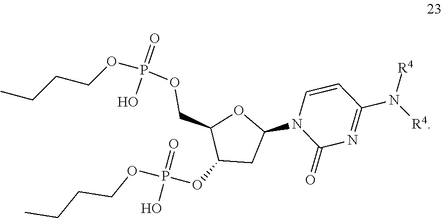

18. The method of any one of clauses 14-17, wherein contacting the dialcohol of Formula 6 or of Formula 7 with the phosphorus oxychloride comprises:

dissolving the dialcohol of Formula 6 or of Formula 7 in a mixture of trialkyl phosphate and phosphorus oxychloride;

stirring the mixture at a temperature from about −20° C. to about 20° C. for a period of time from about 10 minutes to about 3 hours;

adding the alcohol of formula HO(CH₂)ₙCH₃ or HO(CH₂)ₙOH to the mixture; and

stirring the mixture at a temperature from about −20° C. to about 20° C. for a period of time from 1 hour to 10 hours.

19. The method of any one of clauses 14-18, wherein the alcohol of formula HO(CH₂)ₙCH₃ is butanol.

20. The method of any one of clauses 14-18, wherein the alcohol of formula HO(CH₂)ₙOH is 1,4-butanediol.

21. The method of any one of clauses 14-18 or 20, wherein the dialcohol of Formula 6 is a dialcohol of Formula 17

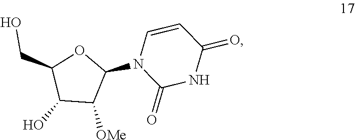 [SMILES: CO[C@@H]1[C@H](O)[C@@H](CO)O[C@H]1n1ccc(=O)[nH]c1=O]

and the Bisphosphocin of Formula 1 is a Bisphosphocin of Formula 10

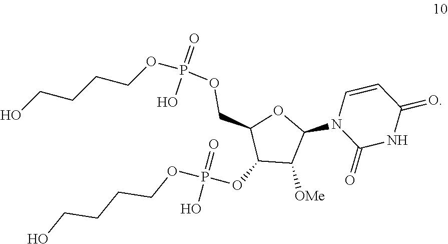 [SMILES: CO[C@@H]1[C@H](OP(=O)(O)OCCCCO)[C@@H](COP(=O)(O)OCCCCO)O[C@H]1n1ccc(=O)[nH]c1=O]

22. The method of any one of clauses 14-19, wherein the dialcohol of Formula 6 is a dialcohol of Formula 18

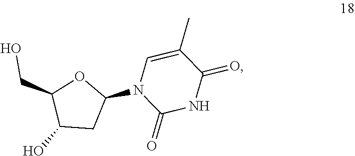 [SMILES: Cc1cn([C@H]2C[C@H](O)[C@@H](CO)O2)c(=O)[nH]c1=O]

and the Bisphosphocin of Formula 1 is a Bisphosphocin of Formula 11

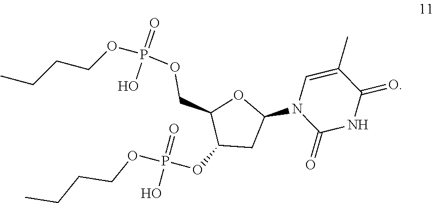 [SMILES: CCCCOP(=O)(O)OC[C@H]1O[C@@H](n2cc(C)c(=O)[nH]c2=O)C[C@@H]1OP(=O)(O)OCCCC]

23. The method of any one of clauses 14-19, wherein the dialcohol of Formula 6 is a dialcohol of Formula 19

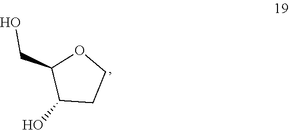

and the Bisphosphocin of Formula 1 is a Bisphosphocin of Formula 12

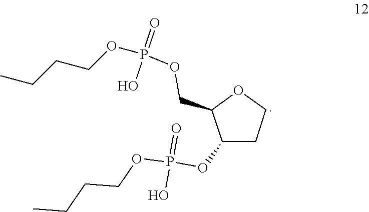 [SMILES: CCCCOP(=O)(O)OC[C@H]1OCC[C@@H]1OP(=O)(O)OCCCC]

24. The method of any one of clauses 14-19, wherein the dialcohol is a dialcohol of Formula 7

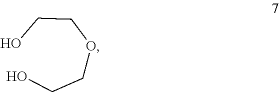

and the Bisphosphocin of Formula 2 is a Bisphosphocin of Formula 13

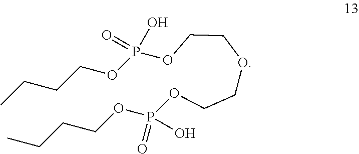 [SMILES: CCCCOP(=O)(O)OCCOCCOP(=O)(O)OCCCC]

25. The method of any one of clauses 14-19, wherein the dialcohol of Formula 6 is a dialcohol of Formula 22

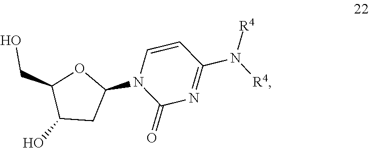

and the Bisphosphocin of Formula 1 is a Bisphosphocin of Formula 23

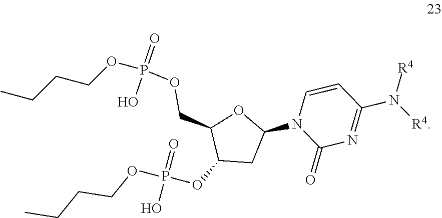

wherein each R⁴ is independently hydrogen, benzyloxycarbonyl, trichloroethoxycarbonyl, t-butoxycarbonyl, benzoyl, acetyl, and 9-fluorenylmethoxycarbonyl.

26. The method of clause 25, further comprising deprotecting the Bisphosphocin of Formula 23

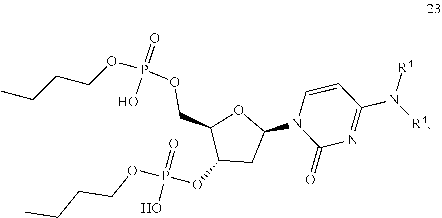

thereby producing a Bisphosphocin of Formula 14:

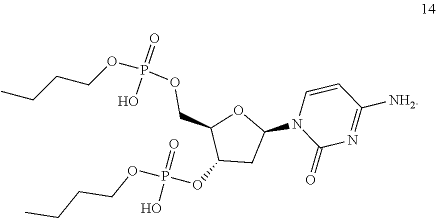 [SMILES: CCCCOP(=O)(O)OC[C@H]1O[C@@H](n2ccc(N)nc2=O)C[C@@H]1OP(=O)(O)OCCCC]

### EXAMPLES

Examples related to the present disclosure are described below. In most cases, alternative techniques can be used. The examples are intended to be illustrative and are not limiting or restrictive of the scope of the invention as set forth in the claims.

### Example 1

### Preparation of Disodium (2R,3S)-2-((Butoxy(Hydroxy)Phosphoryloxy)Methyl)-5-(5-Methyl-2,4-Dioxo-3,4-Dihydropyrimidin-1(2H)-Yl)Tetrahydrofuran-3-Yl Butyl Phosphate

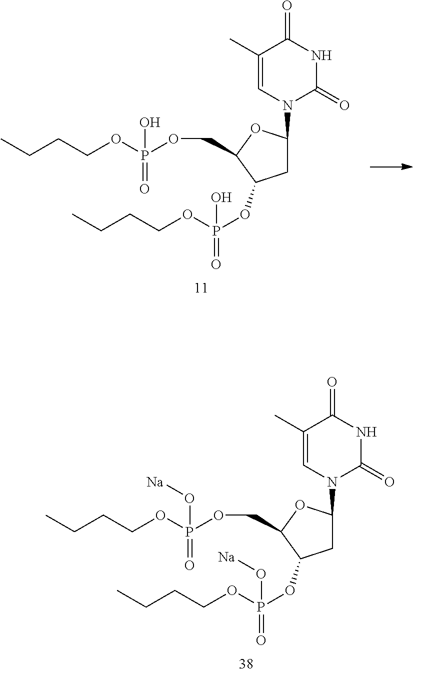

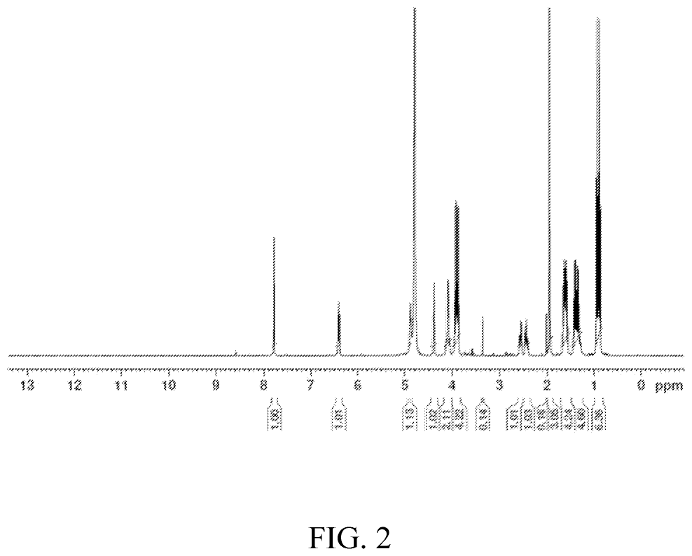

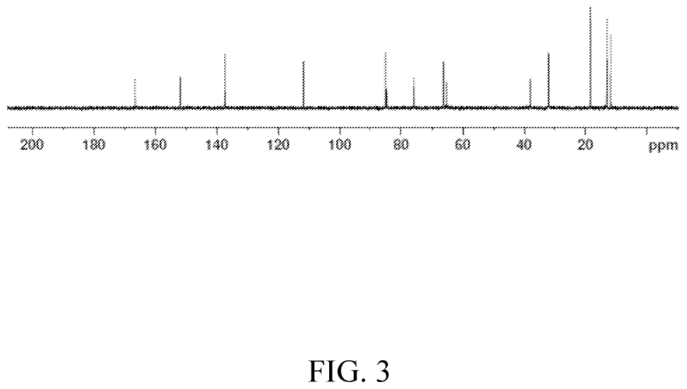

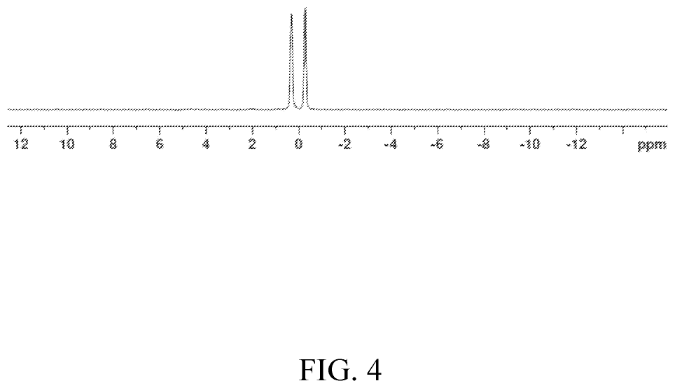

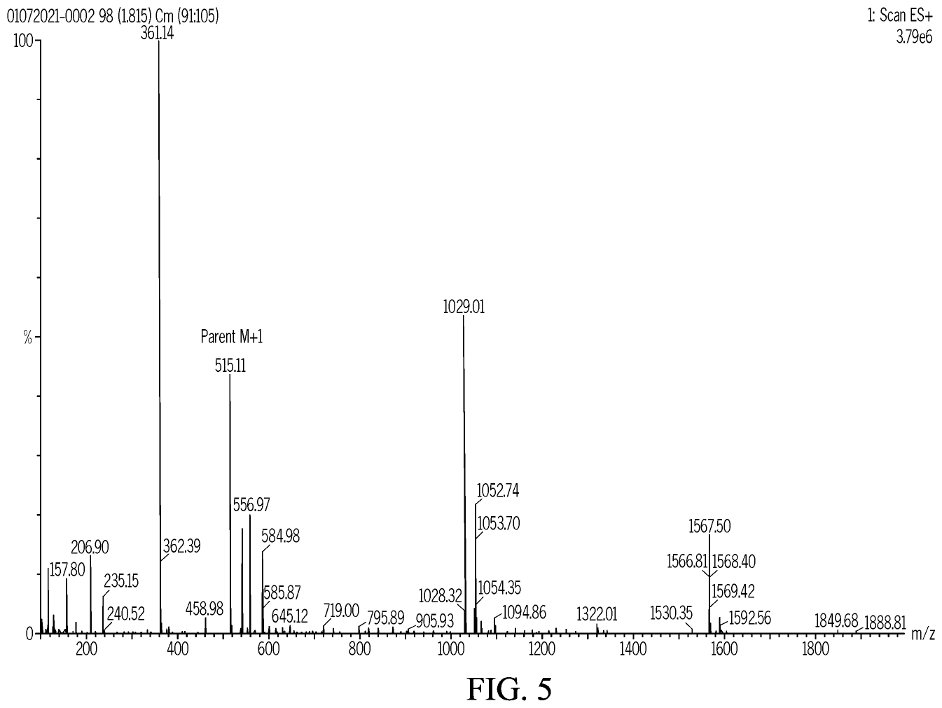

A flask was charged with 400 ml of water and 100 g (195 mmol, 1 equivalent) of (2R,3S)-2-((butoxy(hydroxy)phosphoryloxy)methyl)-5-(5-methyl-2,4-dioxo-3,4-dihydropyr-imidin-1(2H)-yl)tetrahydrofuran-3-yl butyl phosphonate 11 was added and the solution stirred at 0° C. A solution of 15.5 g (387 mmol, 2 equivalents) of sodium hydroxide in 400 ml of water was added slowly to the solution in the flask and the solution warmed to 30° C. The solution was evaporated under vacuum and the resulting solid mass treated four times with 500 ml of isopropyl alcohol, removing the solvent by evaporation under vacuum each time. The solid cake was then slurried with 500 ml of heptane, and the slurry was filtered under a dry, inert atmosphere and dried to produce 53 g (48% yield) of final product. IR spectrum peaks: 3440, 2961, 1699, 1476, 1434, 1384, 1279, 1222, 1091, 1069, 1031, 972, 897, 836, 784, 733, 616, 561 cm⁻¹. FIG. 2 provides the ¹H NMR spectrum of compound 38. FIG. 3 provides the ¹³C NMR spectrum of compound 38. FIG. 4 provides the ³¹P NMR spectrum of compound 38. FIG. 5 provides the mass spectrum of compound 38. IR spectrum peaks: 3440, 2961, 1699, 1476, 1434, 1384, 1279, 1222, 1091, 1069, 1031, 972, 897, 836, 784, 733, 616, 561 cm⁻¹.

### Example 2

### Preparation of Disodium (2R,3S,5R)-5-(4-Amino-2-Oxopyridin-1(2H)-Yl)-3-(Butoxy(Hydroxy)Phosphoryloxy)Tetrahydrofuran-2-Yl)Methyl Butyl Phosphate

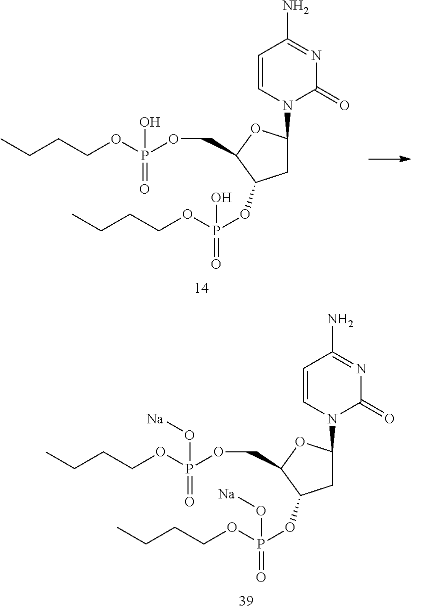

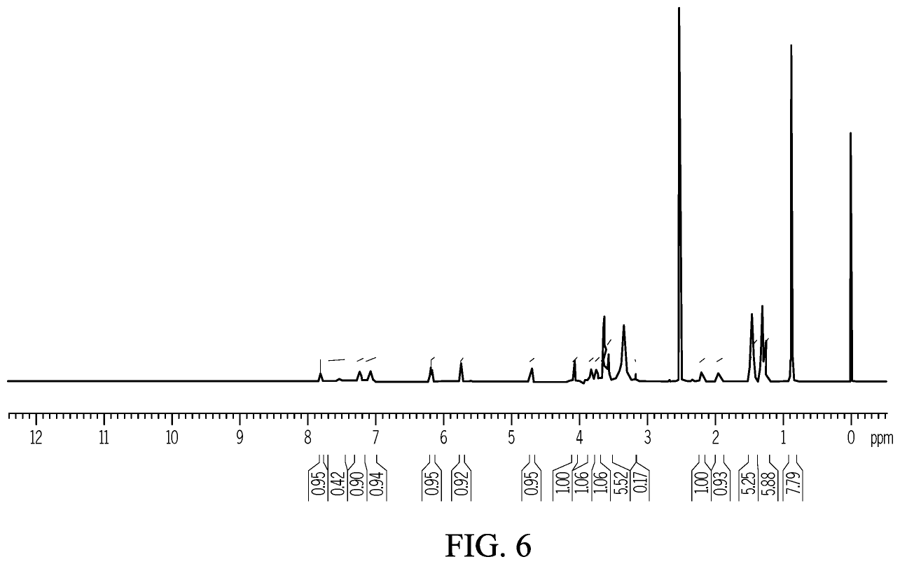

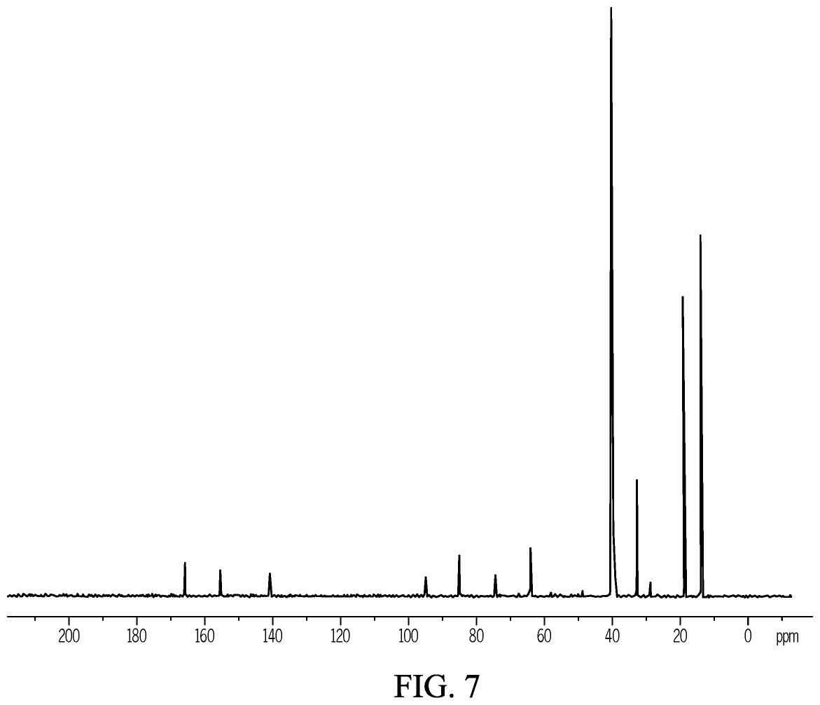

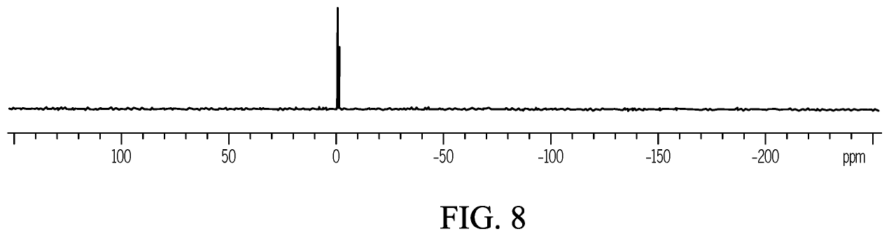

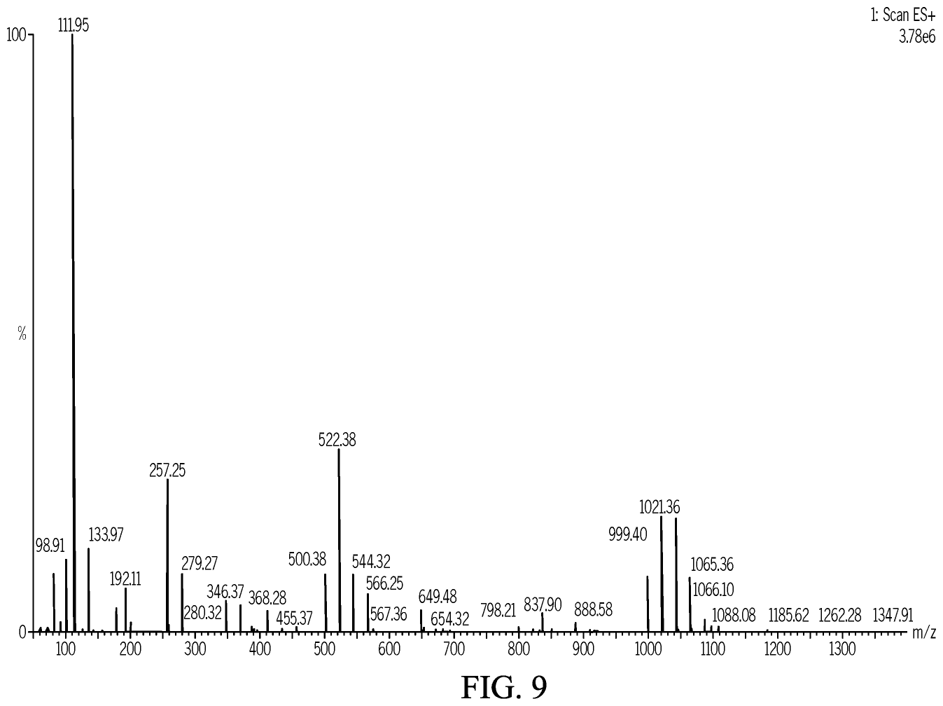

A flask was charged with 80 ml of water and 16 g (32 mmol, 1 equivalent) of (2R,3S,5R)-5-(4-amino-2-oxopyridin-1(2H)-yl)-3-(butoxy(hydroxy)phosphoryloxy)tetrahy-drofuran-2-yl)methyl butyl phosphonate 14 was added and the solution stirred at 0° C. A solution of 2.6 g (64 mmol, 2 equivalents) of sodium hydroxide in 80 ml of water was added slowly to the solution in the flask and the solution warmed to 30° C. The solution was evaporated under vacuum and the resulting solid mass treated four times with 100 ml of isopropyl alcohol, removing the solvent by evaporation under vacuum each time. The solid cake was then slurried with 100 ml of heptane, and the slurry was filtered under a dry, inert atmosphere and dried to produce 15 g (86% yield) of final product 39. IR spectrum peaks: 3411, 2961, 1655, 1528, 1497, 1293, 1219, 1091, 1069, 1031, 976, 897, 826, 729, 562 cm⁻¹. FIG. 6 provides the ¹H NMR spectrum of compound 39. FIG. 7 provides the ¹³C NMR spectrum of compound 39. FIG. 8 provides the ³¹P NMR spectrum of compound 39. FIG. 9 provides the mass spectrum of compound 39. IR spectrum peaks: 3411, 2961, 1655, 1528, 1497, 1293, 1219, 1091, 1069, 1031, 976, 897, 826, 729, 562 cm⁻¹.

### Example 3

### Preparation of (2R,3S,5R)-5-(4-Amino-2-Oxopyridin-1(2H)-Yl)-3-(Butoxy(Hydroxy)Phosphoryloxy)Tetrahydrofuran-2-Yl)Methyl Butyl Phosphonate

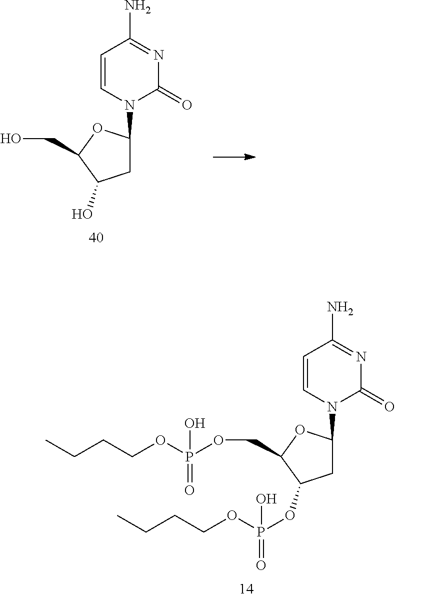

A flask was charged with 10 ml of triethyl phosphate and 2 g (13.2 mmol, 3 equivalents) of phosphorous oxychloride and mixed. The mixture was cooled to 0° C. and 1 g (4.4 mmol, 1 equivalent) of 2′-deoxycytidine 40 was added, and the mixture stirred at 0° C. After 2 hours, 1.63 g (22 mmol, 5 equivalents) of butanol was added and the mixture was stirred at 0° C. for 5 hours. The reaction mass was quenched with ice-cold water and the pH was adjusted to 7 by the addition of sodium bicarbonate. The aqueous layer was washed with methyl-t-butyl ether to remove water insoluble impurities and the aqueous layer was condensed to produce 0.7 g (32% yield) of product 14. The crude material was passed through a Dowex resin column and the fractions containing the product pooled and evaporated. LCMS data: Retention Time (RT) 1.8 min, 22.59%, M+1=364(monophosphorylated product); RT 3.6 min, 45.85%, M+1=500 (target product 41).

### Example 4

### Preparation of 5-(4-[(Phenylmethoxy)Carbonyl]-Amino-2-Oxopyridin-1(2H)-Yl)-3-(Butoxy(Hydoxy)Phosphoryloxy)Tetrahydrofuran-2-Yl)Methyl Butyl Phosphonate

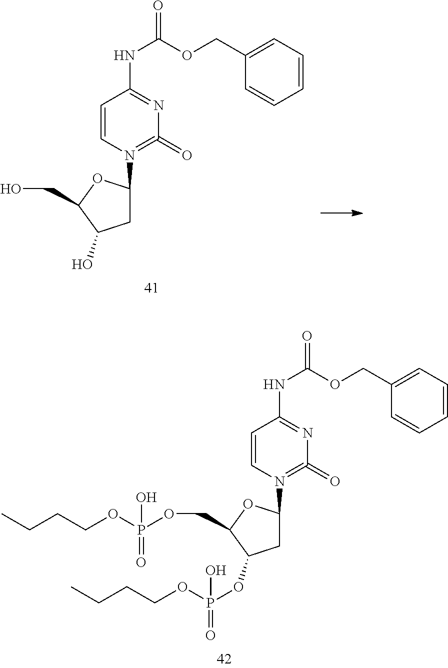

A flask was charged with 50 ml of triethyl phosphate and 13 g (83 mmol, 3 equivalents) of phosphorous oxychloride and mixed. The mixture was cooled to 0° C. and 10 g (27.7 mmol, 1 equivalent) of benzyloxycarbonyl-protected 2′-deoxycytidine 41 was added, and the mixture stirred at 0° C. After 2 hours 10.3 g (138.5 mmol, 5 equivalents) of butanol was added and the mixture was stirred at 0° C. for 5 hours. The reaction mass was quenched with ice-cold water and the aqueous layer was washed with ethyl acetate to remove water insoluble impurities and the aqueous layer was condensed to produce 11.75 g (67% yield) of product 42. The crude material was passed through a Dowex resin column and the fractions containing the product pooled and evaporated. LCMS data: Retention Time (RT) 1.6 min, 14.4%, M+1=500 (product 14); RT 1.9 min, 39.2%, M+1=183 (EtO₃P═O); RT 2.0 min, 33.2%, M+1=634, target product 42).

### Example 5

### Preparation of Bis-2,2,2-Trifluoroethyl Butyl Phosphate

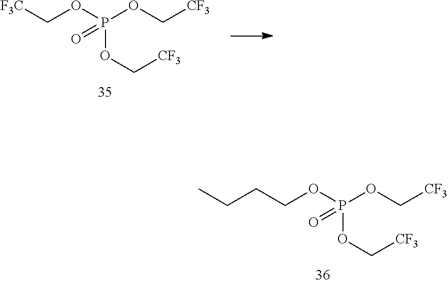 [SMILES: O=P(OCC(F)(F)F)(OCC(F)(F)F)OCC(F)(F)F ; CCCCOP(=O)(OCC(F)(F)F)OCC(F)(F)F]

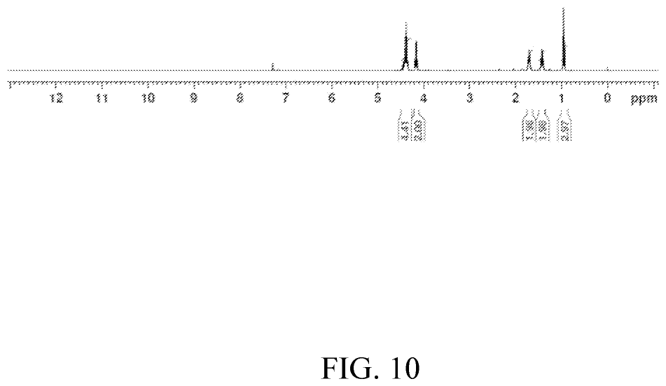

A flask was charged with 750 ml of toluene and to it added 278.5 g (810 mmol, 1.2 equivalents)tris-trifluoroethyl phosphate 35. The solution was cooled to 0° C. and 103 g (675 mmol, 1 equivalent) of 1,8-diazabicyclo[5.4.0]undec-7-ene was added, followed by 50 g (675 mmol, 1 equivalent) of butanol. The reaction mixture was allowed to warm to room temperature. The mixture was quenched by the addition of phosphate buffer (pH 7) at 0° C. The mixture was extracted twice with ethyl acetate. The combined extracts were washed with aqueous brine, dried over sodium sulphate, and concentrated in vacuo to yield 210 g (98% yield) of bis-trifluoroethyl butyl phosphate 36. FIG. 10 provides the ¹H NMR spectrum of compound 36. LCMS data: Retention Time (RT) 6.19 min, 99.6%, M+1=319 (target product 36).

### Example 6

### Preparation of 2,2,2-Trifluoroethy Butyl Prop-2-Enyl Phosphate

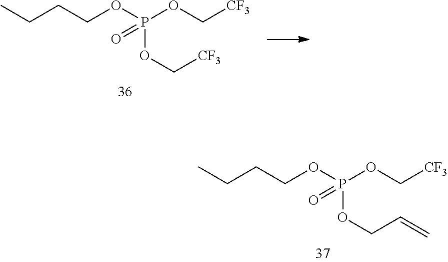 [SMILES: CCCCOP(=O)(OCC(F)(F)F)OCC(F)(F)F ; C=CCOP(=O)(OCCCC)OCC(F)(F)F]

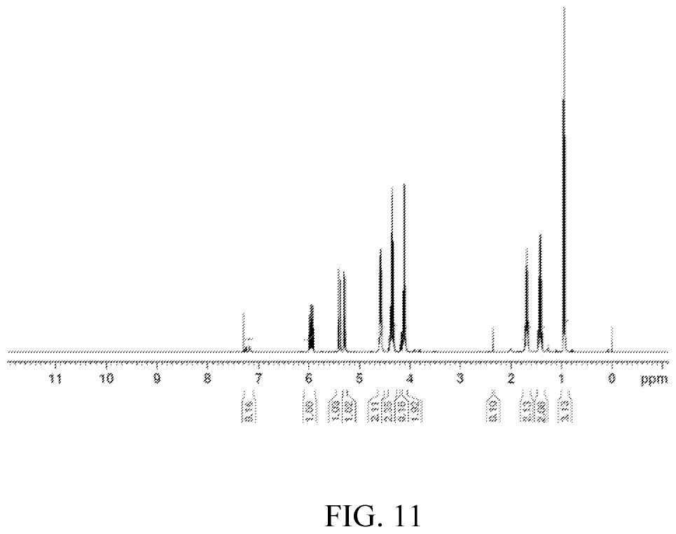

A flask was charged with 50 g (157 mmol, 1 equivalent) of bis-trifluoroethyl butyl phosphate 36 in 750 ml of toluene and cooled to −30° C. To the cooled solution was added 71.7 g (471 mmol, 3 equivalents) of 1,8-diazabicyclo[5.4.0]undec-7-ene and 9.12 g (157 mmol, 1 equivalent) of allyl alcohol. The reaction mixture was allowed to come to room temperature overnight. The mixture was quenched by the addition of phosphate buffer (pH 7) at 0° C. The mixture was extracted twice with methyl t-butyl ether. The combined extracts were washed with aqueous brine, dried over sodium sulphate, and concentrated in vacuo to yield 33 g (76% yield) of 2,2,2-trifluoroethyl butyl prop-2-enyl phosphate 37. FIG. 11 provides the ¹H NMR spectrum of compound 37. LCMS data: Retention Time (RT) 2.81 min, 46.4%, M+1=277 (target product 37).

### Example 7

### Preparation of 5-(4-[(Phenylmethoxy)Carbonyl]-Amino-2-Oxopyridin-1(2H)-Yl)-3-(Butoxy(Prop-2-Enyloxy)Phosphoryloxy)Tetrahydrofuran-2-Yl)Methyl Butyl-Prop-2-Enyl Phosphate

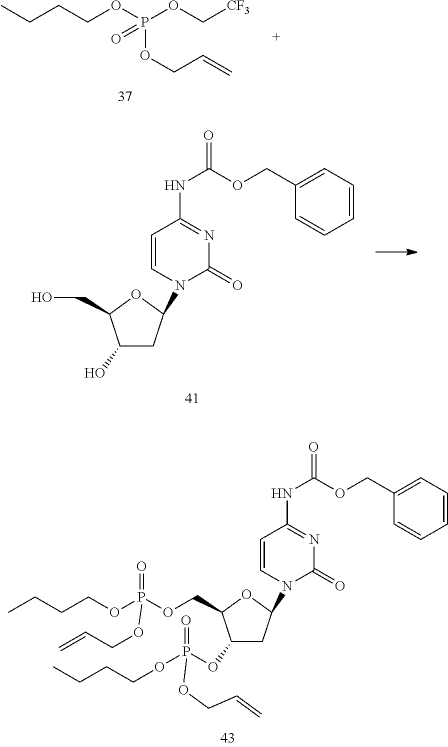 [SMILES: C=CCOP(=O)(OCCCC)OCC(F)(F)F ; O=C(Nc1ccn([C@H]2C[C@H](O)[C@@H](CO)O2)c(=O)n1)OCc1ccccc1 ; C=CCOP(=O)(OCCCC)OC[C@H]1O[C@@H](n2ccc(NC(=O)OCc3ccccc3)nc2=O)C[C@@H]1OP(=O)(OCC=C)OCCCC]

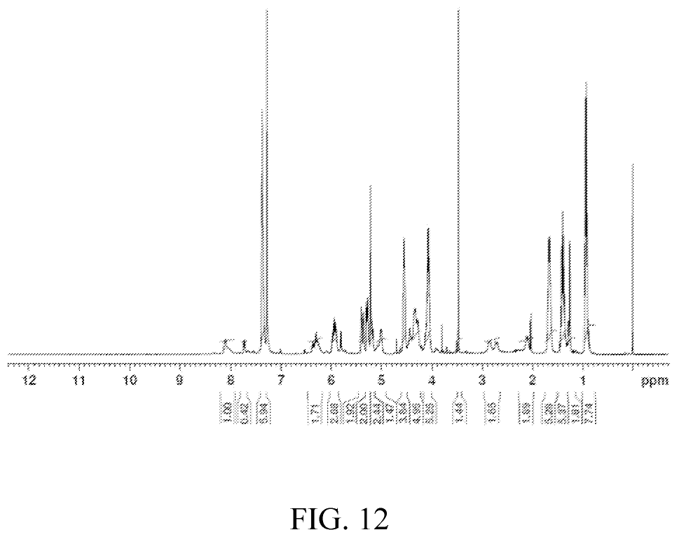

A flask was charged with 5 g (13.8 mmol, 1 equivalent) of 2′-deoxy-N-[(phenylmethoxy)carbonyl]-cytidine phosphate 41 in 50 ml of THE and cooled to −40° C. To the cooled solution was added 10.1 g (55.2 mmol, 4 equivalents) of sodium hexamethyldisilazide followed by 19 g (55.2 mmol, 5 equivalents) of 2,2,2-trifluoroethyl butyl prop-2-enyl phosphate 37. The reaction mixture was allowed to come to room temperature overnight. The mixture was quenched by the addition of phosphate buffer (pH 7) at 0° C. The mixture was extracted twice with methyl t-butyl ether. The combined extracts were washed with aqueous brine, dried over sodium sulphate, and concentrated in vacuo to yield 3.9 g (39% yield) of (2R,3S,5R)-5-(4-[(phenylmethoxy)carbonyl]-amino-2-oxopyridin-1(2H)-yl)-3-(butoxy-(prop-2-enyloxy)phosphoryloxy)tetrahydrofuran-2-yl)methyl butyl-prop-2-enyl phosphate 43. FIG. 12 provides the ¹H NMR spectrum of compound 43. LCMS data: Retention Time (RT) 2.97 min, 28%, M+1=714 (target product 43).

### Example 8

### Preparation of 5-(4-[(Phenylmethoxy)Carbonyl]-Amino-2-Oxopyridin-1(2H)-Yl)-3-(Butoxy(Hydoxy)Phosphoryloxy)Tetrahydrofuran-2-Yl)Methyl Butyl Phosphonate

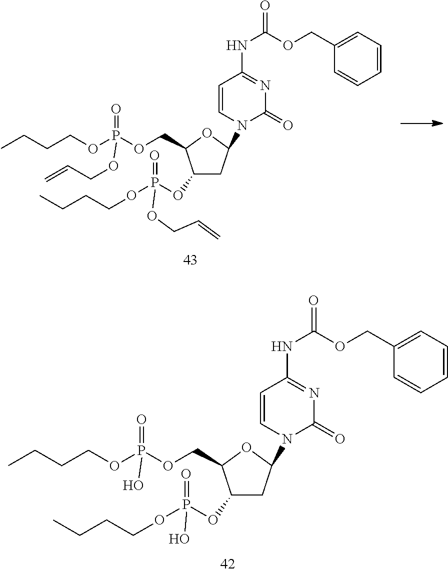 [SMILES: C=CCOP(=O)(OCCCC)OC[C@H]1O[C@@H](n2ccc(NC(=O)OCc3ccccc3)nc2=O)C[C@@H]1OP(=O)(OCC=C)OCCCC ; CCCCOP(=O)(O)OC[C@H]1O[C@@H](n2ccc(NC(=O)OCc3ccccc3)nc2=O)C[C@@H]1OP(=O)(O)OCCCC]

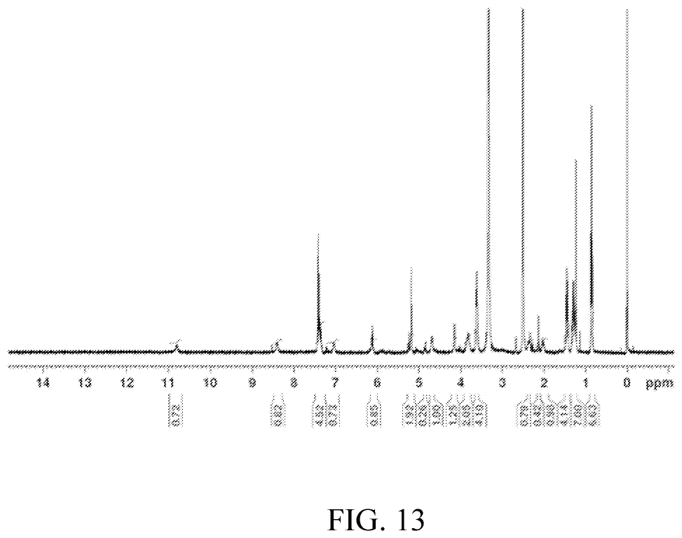

A flask was charged with a solution of 0.4 g (0.56 mmol, 1 equivalent) 43 in 4 ml of acetone and 0.25 g (1.7 mmol, 3 equivalents) of sodium iodide was added, and the solution heated at reflux for 3 h. The solution was diluted with 10 ml water and extracted with 20 ml of methyl t-butyl ether. The aqueous layer was lyophilized to yield 0.27 g (62% yield) of the product 42. FIG. 13 provides the ¹H NMR spectrum of compound 42 prepared according to this method. LCMS data: Retention Time (RT) 1.97 min, 62.3%, M+1=634 (target product 42).

### Example 9

### Preparation of (2R,3S,5R)-5-(4-Amino-2-Oxopyridin-1(2H)-Yl)-3-(Butoxy(Hydroxy)Phosphoryloxy)Tetrahydrofuran-2-Yl)Methyl Butyl Phosphonate

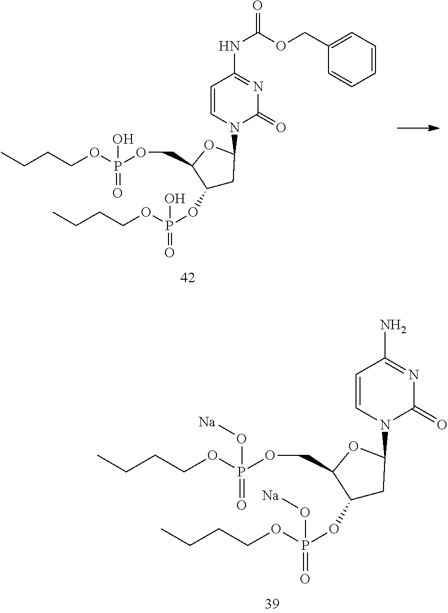

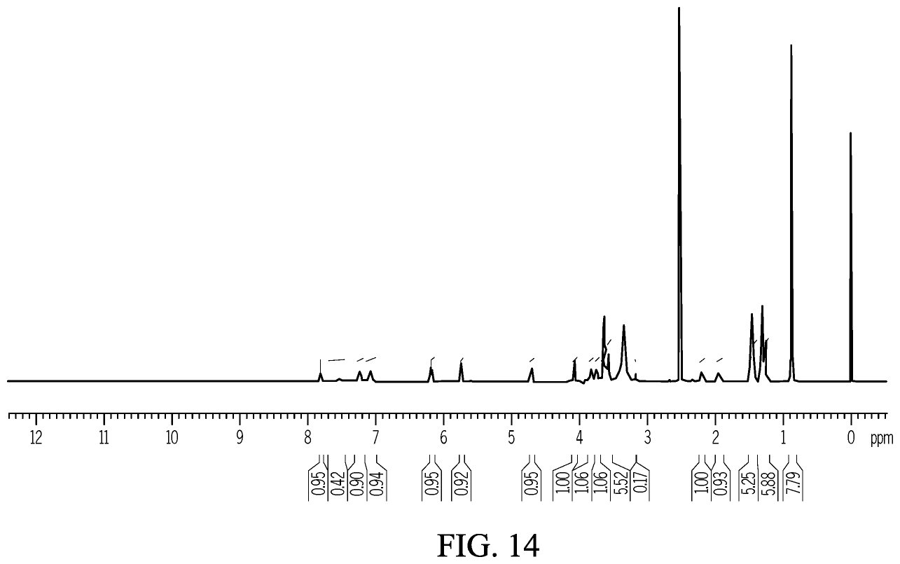

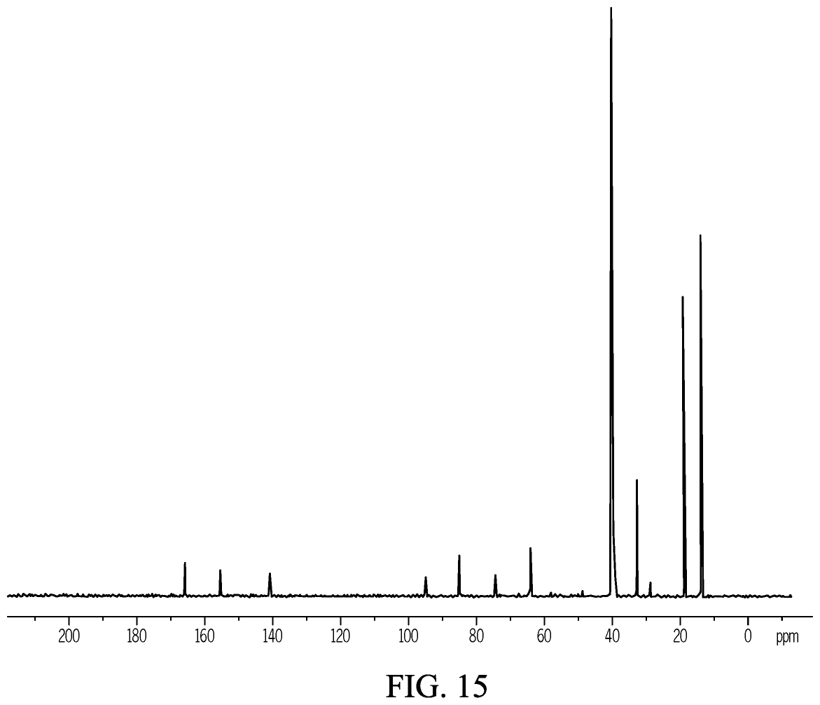

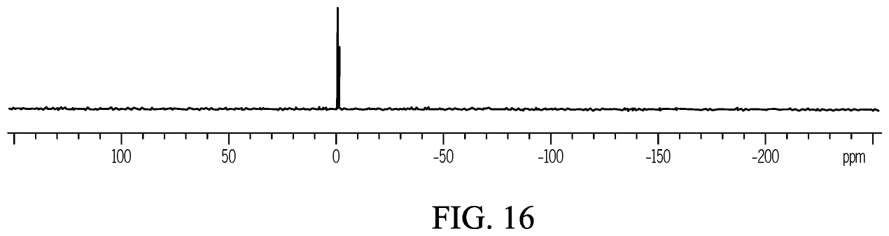

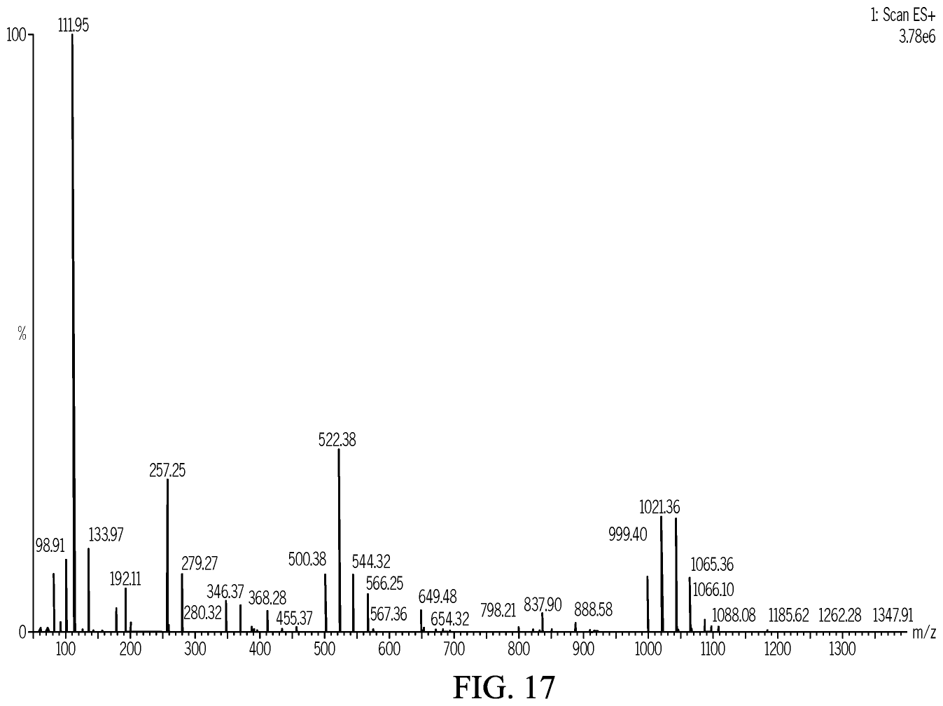

A flask was charged with a solution of 4 g (6.3 mmol, 1 equivalent) of 5-(4-[(phenylmethoxy)carbonyl]-amino-2-oxopyridin-1(2H)-yl)-3-(butoxy(hydoxy)phosphoryl-oxy)tetrahydrofuran-2-yl)methyl butyl phosphonate 42 in 20 ml of ethanol and a solution of 0.88 g (18 mmol, 3.5 equivalents) of sodium hydroxide in 1.6 ml of water was added. The reaction mixture was heated to 45° C. and stirred for 24 hours. The turbid suspension was concentrated to 5 ml and the aqueous layer was washed three times with 10 ml of methyl-t-butyl ether to remove the benzyl alcohol. The final solution was evaporated and slurried with heptane to produce 2.4 g (70% yield) of the product 39. FIG. 14 provides the ¹H NMR spectrum of compound 39. FIG. 15 provides the ¹³C NMR spectrum of compound 39. FIG. 16 provides the ³¹P NMR spectrum of compound 39. FIG. 17 provides the mass spectrum of compound 39.

IR spectrum peaks: 3411, 2961, 1655, 1528, 1497, 1293, 1219, 1091, 1069, 1031, 976, 897, 826, 729, 562 cm⁻¹.

### Example 10

### Preparation of 5-(4-[(Phenylmethoxy)Carbonyl]-Amino-2-Oxopyridin-1(2H)-Yl)-3-(butoxy(2,2,2-trifluoroethylprop-2-enyloxy)phosphoryloxy)tetrahydrofuran-2-yl)methyl butyl-2,2,2-trifluoroethyl phosphate

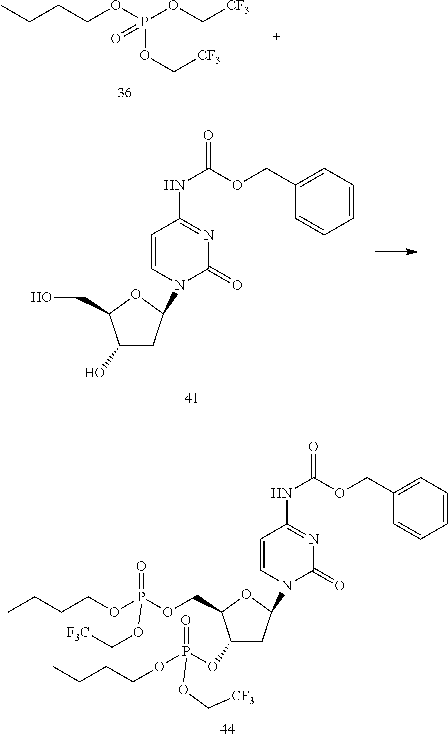 [SMILES: CCCCOP(=O)(OCC(F)(F)F)OCC(F)(F)F ; O=C(Nc1ccn([C@H]2C[C@H](O)[C@@H](CO)O2)c(=O)n1)OCc1ccccc1 ; CCCCOP(=O)(OC[C@H]1O[C@@H](n2ccc(NC(=O)OCc3ccccc3)nc2=O)C[C@@H]1OP(=O)(OCCCC)OCC(F)(F)F)OCC(F)(F)F]

A flask was charged with 1 g (2.76 mmol, 1 equivalent) of 2′-deoxy-N-[(phenylmethoxy)carbonyl]-cytidine phosphate 41 in 15 ml of THE and cooled to −50° C. To the cooled solution was added 1.52 g (8.3 mmol, 3 equivalents) of sodium hexamethyldisilazide followed by 0.88 g (8.3 mmol, 3 equivalents) of 2,2,2-trifluoroethyl butyl prop-2-enyl phosphate 36. The reaction mixture was allowed to come to room temperature overnight. The mixture was quenched by the addition of phosphate buffer (pH 7) at 0° C. The mixture was extracted twice with methyl t-butyl ether. The combined extracts were washed with aqueous brine, dried over sodium sulphate, and concentrated in vacuo to yield 2 g (75% yield) of 5-(4-[(phenylmethoxy)carbonyl]-amino-2-oxopyridin-1(2H)-yl)-3-(butoxy(2,2,2-trifluoroethyl-prop-2-enyloxy)phosphoryloxy)tetrahydrofuran-2-yl)methyl butyl-2,2,2-trifluoroethyl phosphate 44. LCMS data: Retention Time (RT) 6.08 min, 82.6%, M+1=798 (target product 44).

### Example 11

### Preparation of (2R,3S,5R)-5-(4-Amino-2-Oxopyridin-1(2H)-Yl)-3-(Butoxy(Hydroxy)Phosphoryloxy)Tetrahydrofuran-2-Yl)Methyl Butyl Phosphonate

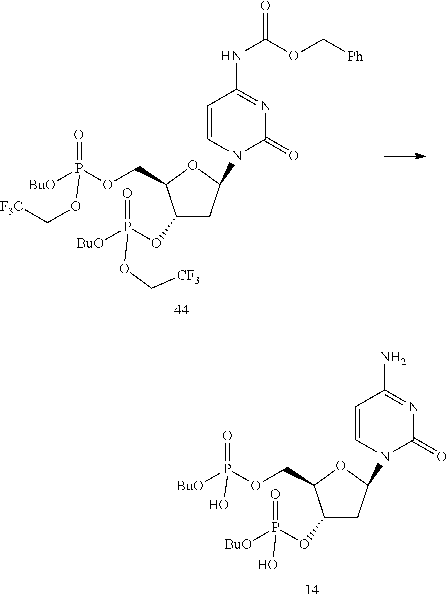

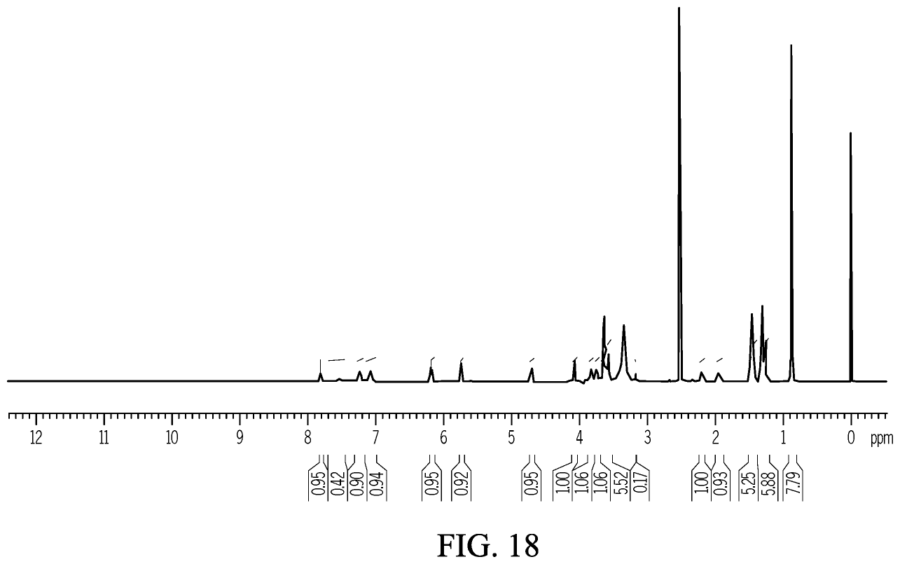

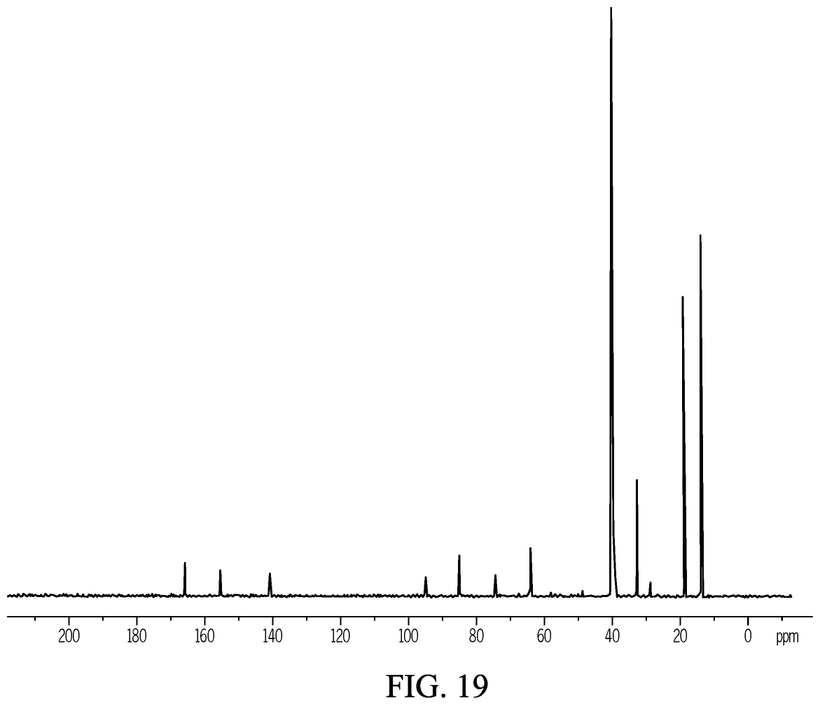

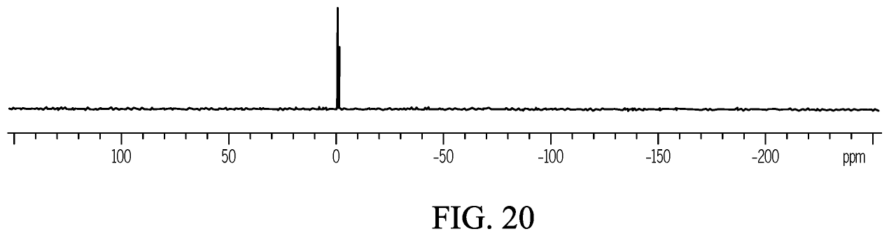

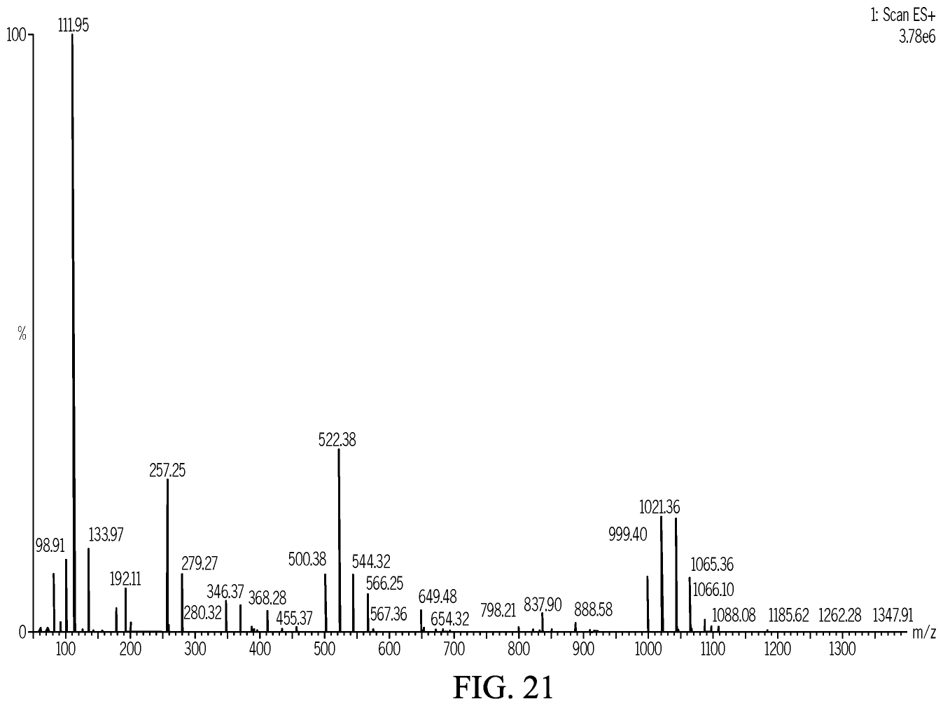

A flask was charged with a solution of 0.5 g (0.62 mmol, 1 equivalent) of 5-(4-[(phenylmethoxy)carbonyl]-amino-2-oxopyridin-1(2H)-yl)-3-(butoxy(2,2,2-trifluoroethyl-prop-2-enyloxy)phosphoryloxy)tetrahydrofuran-2-yl)methyl butyl-2,2,2-trifluoroethyl phosphate 44 in 2.5 ml of water and a solution of 0.26 g (1.5 mmol, 2.5 equivalents) of barium hydroxide was added. The reaction mixture was heated to 55° C. and stirred for 20 hours. The reaction mixture contained 41% of the product 14. FIG. 18 provides the ¹H NMR spectrum of compound 14 prepared by this method. FIG. 19 provides the ¹³C NMR spectrum of compound 14 prepared by this method. FIG. 20 provides the ³¹P NMR spectrum of compound 14 prepared by this method. FIG. 21 provides the mass spectrum of compound 14 prepared by this method. IR spectrum peaks: 3411, 2961, 1655, 1528, 1497, 1293, 1219, 1091, 1069, 1031, 976, 897, 826, 729, 562 cm⁻¹.

### Example 12

### Preparation of 3′,5′-Bis-Tertiarybutyldimethylsilyl-2′-Deoxycytidine

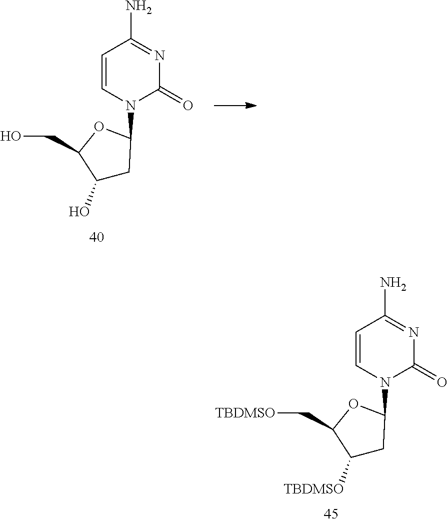 [SMILES: Nc1ccn([C@H]2C[C@H](O)[C@@H](CO)O2)c(=O)n1 ; CC(C)(C)[Si](C)(C)OC[C@H]1O[C@@H](n2ccc(N)nc2=O)C[C@@H]1O[Si](C)(C)C(C)(C)C]

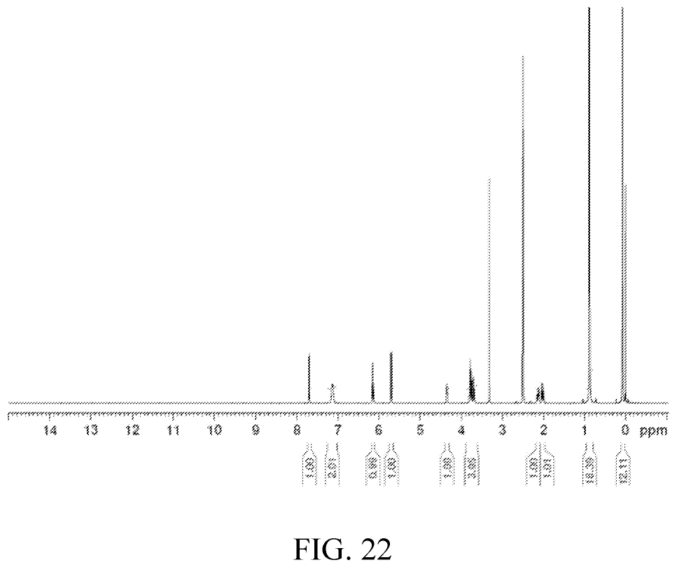

A flask was charged with 1,000 ml of DMF and 200 g (0.88 mol, 1 equivalent) of 2′-deoxycytidine 40. The solution was cooled to 5° C. and 209 g (3.1 mol, 3.5 equivalents) of imidazole was added. Maintaining the temperature at 5° C., 400 g (2.64 mol, 3 equivalents) of tertiarybutyldimethylsilyl chloride was slowly added. The reaction was quenched by the addition of and extract with MTBE/heptane slurry to give 320 g (80% yield) of 3′,5′-bis-tertiarybutyldimethylsilyl-2′-deoxycytidine 45. FIG. 22 provides the ¹H NMR spectrum of compound 45. LCMS data: Retention Time (RT) 16.2 min, 97.3%, M+1=456 (target product 45).

### Example 13

### Preparation of 3′,5′-Bis-Tertiarybutyldimethylsilyl-2′-Deoxy-N-[(Phenylmethoxy)Carbonyl]-Cytidine

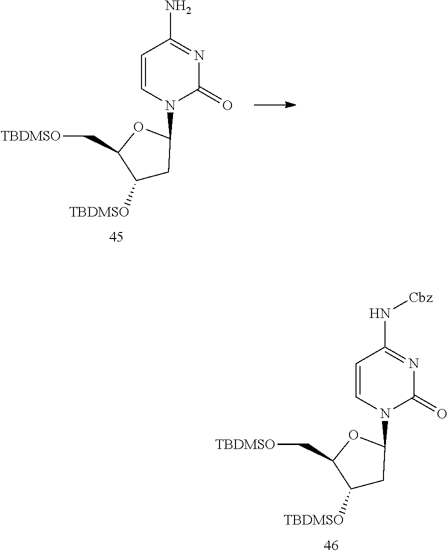 [SMILES: CC(C)(C)[Si](C)(C)OC[C@H]1O[C@@H](n2ccc(N)nc2=O)C[C@@H]1O[Si](C)(C)C(C)(C)C ; CC(C)(C)[Si](C)(C)OC[C@H]1O[C@@H](n2ccc(NC(=O)OCc3ccccc3)nc2=O)C[C@@H]1O[Si](C)(C)C(C)(C)C]

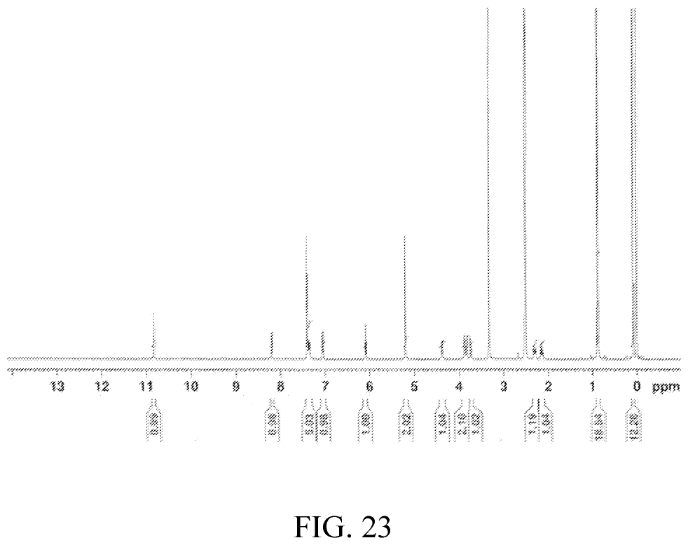

A flask was charged with 3,000 ml of acetonitrile and 600 ml of DMF, followed by 300 g (0.66 mol, 1 equivalent) of 3′,5′-bis-tertiarybutyldimethylsilyl-2′-deoxycytidine 45. The solution was cooled to 5° C. and 161 g (1.32 mol, 2 equivalents) of DMAP was added. Maintaining the temperature at 5° C., 225 g (1.32 mol, 2 equivalents) of phenylmethoxycarbonyl chloride was slowly added. Quench and extract with MTBE, heptane slurry to give 320 g (82% yield) of 3′,5′-bis-tertiarybutyldimethylsilyl-2′-deoxy-N-[(phenylmethoxy)carbonyl]-cytidine 46. FIG. 23 provides the ¹H NMR spectrum of compound 46. LCMS data: Retention Time (RT) 18.7 min, 100%, M+1=590 (target product 46).

### Example 14

### Preparation of 2′-Deoxy-N-[(Phenylmethoxy)Carbonyl]-Cytidine

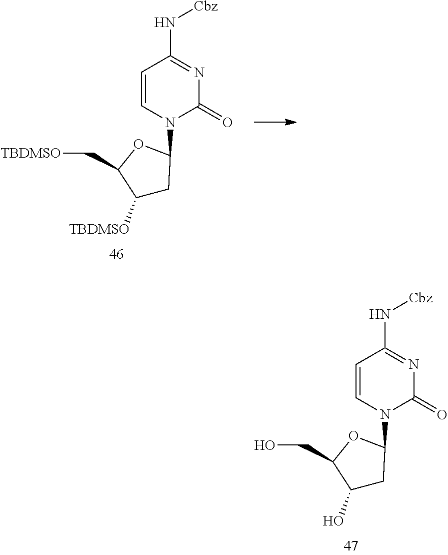 [SMILES: CC(C)(C)[Si](C)(C)OC[C@H]1O[C@@H](n2ccc(NC(=O)OCc3ccccc3)nc2=O)C[C@@H]1O[Si](C)(C)C(C)(C)C ; O=C(Nc1ccn([C@H]2C[C@H](O)[C@@H](CO)O2)c(=O)n1)OCc1ccccc1]

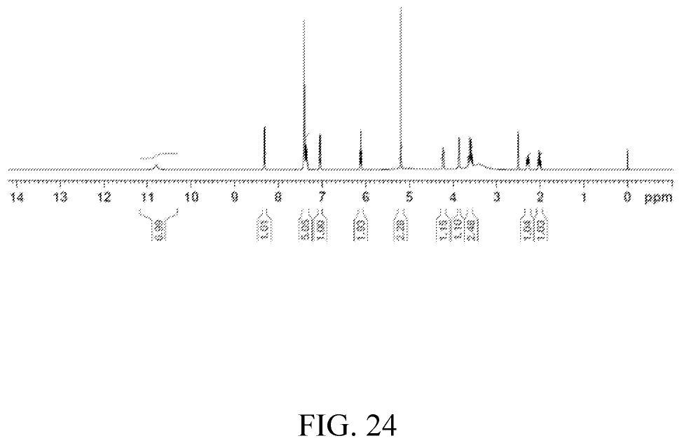

A flask was charged with 170 ml of methanol followed by 24.4 g (38 mmol, 1 equivalent) of 3′,5′-bis-tertiarybutyldimethylsilyl-2′-deoxy-N-[(phenylmethoxy)carbonyl]-cytidine 46 and the resulting solution cooled to 5° C. To the solution was added 24.4 ml of concentrated hydrochloric acid over 45 minutes maintaining the temperature at 5° C., and the solution stirred at 5° C. for 5 hours. The reaction was then quenched by the slow addition of 270 ml of 10% sodium bicarbonate solution in water maintaining the temperature at 10° C. (the reaction liberates CO₂ gas). After the heterogeneous mixture was allowed to warm to room temperature, the solid product was filtered off and washed with water. The filtered solid was slurried with a mixture of 200 ml of 4:1 heptane-ethyl acetate mixture and filtered and washed with 50 ml of the 4:1 heptane-ethyl acetate mixture and finally dried in a vacuum oven to give 11.3 g (82% yield) of the product 47. FIG. 24 provides the ¹H NMR spectrum of compound 47. LCMS data: Retention Time (RT) 10.6 min, 99.4%, M+1=362 (target product 47).

### Example 15

### Preparation of 5-(4-Amino-2-Oxopyridin-1(2H)-Yl)-3-(Butoxy(2,2,2-Trifluoroethylprop-2-Enyloxy)Phosphoryloxy)Tetrahydrofuran-2-Yl)Methyl Butyl-2,2,2-Trifluoroethyl Phosphate

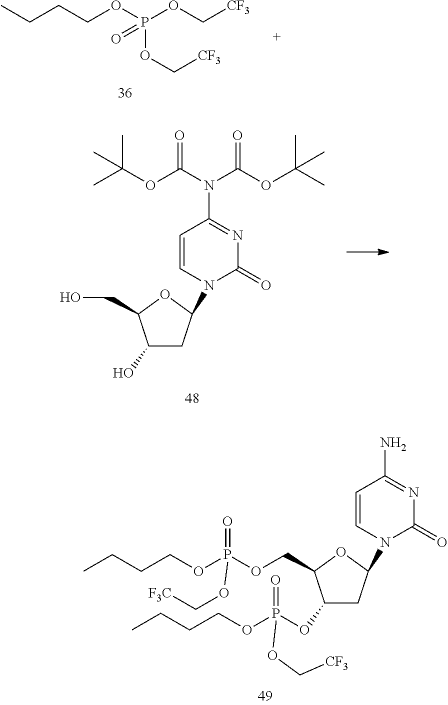 [SMILES: CCCCOP(=O)(OCC(F)(F)F)OCC(F)(F)F ; CC(C)(C)OC(=O)N(C(=O)OC(C)(C)C)c1ccn([C@H]2C[C@H](O)[C@@H](CO)O2)c(=O)n1 ; CCCCOP(=O)(OC[C@H]1O[C@@H](n2ccc(N)nc2=O)C[C@@H]1OP(=O)(OCCCC)OCC(F)(F)F)OCC(F)(F)F]

A flask was charged with 5 g (11.7 mmol, 1 equivalent) of 2′-deoxy-N-[bis(tertiary butyloxycarbonyl)]-cytidine 48 in 50 ml of THF and cooled to −45° C. To the cooled solution was added 8.6 g (46.8 mmol, 4 equivalents) of sodium hexamethyldisilazide and 14.9 g (46.8 mmol, 4 equivalents) of 2,2,2-trifluoroethyl butyl prop-2-enyl phosphate 36 and the reaction mixture was stirred at −45° C. for 4 hours. The mixture was quenched by the addition of water at 0° C. The mixture was extracted twice with ethyl acetate and the combined extracts were concentrated under vacuum. The crude product was dissolved in 50 ml dichloromethane and cooled to −40° C. To the stirred solution was added 10 ml of trifluoroacetic acid and the solution was stirred for 2 hours at −40° C. The reaction was quenched with 5% sodium bicarbonate solution and the dichloromethane layer separated, washed with brine, dried over sodium sulphate to yield 7.4 g of 5-(4-amino-2-oxopyridin-1(2H)-yl)-3-(butoxy(2,2,2-trifluoro-ethylprop-2-enyloxy)phosphoryloxy)tetrahydrofuran-2-yl)methyl butyl-2,2,2-trifluoroethyl phosphate 49. LCMS data: Retention Time (RT) 2.41 min, 71.4%, M+1=664 (target product 49).

### Example 16

### Preparation of (2R,3S,5R)-5-(4-Amino-2-Oxopyridin-1(2H)-Yl)-3-(Butoxy(Hydroxy)Phosphoryloxy)Tetrahydrofuran-2-Yl)Methyl Butyl Phosphonate

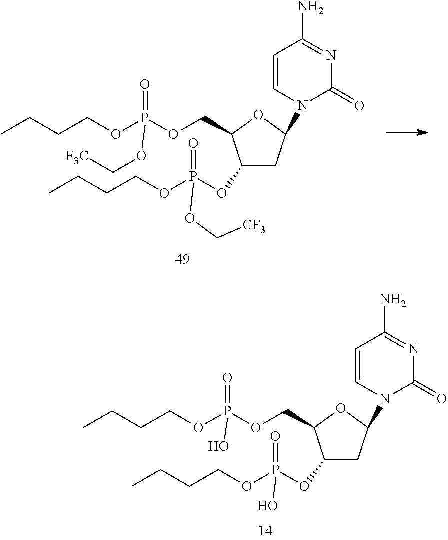 [SMILES: CCCCOP(=O)(OC[C@H]1O[C@@H](n2ccc(N)nc2=O)C[C@@H]1OP(=O)(OCCCC)OCC(F)(F)F)OCC(F)(F)F ; CCCCOP(=O)(O)OC[C@H]1O[C@@H](n2ccc(N)nc2=O)C[C@@H]1OP(=O)(O)OCCCC]

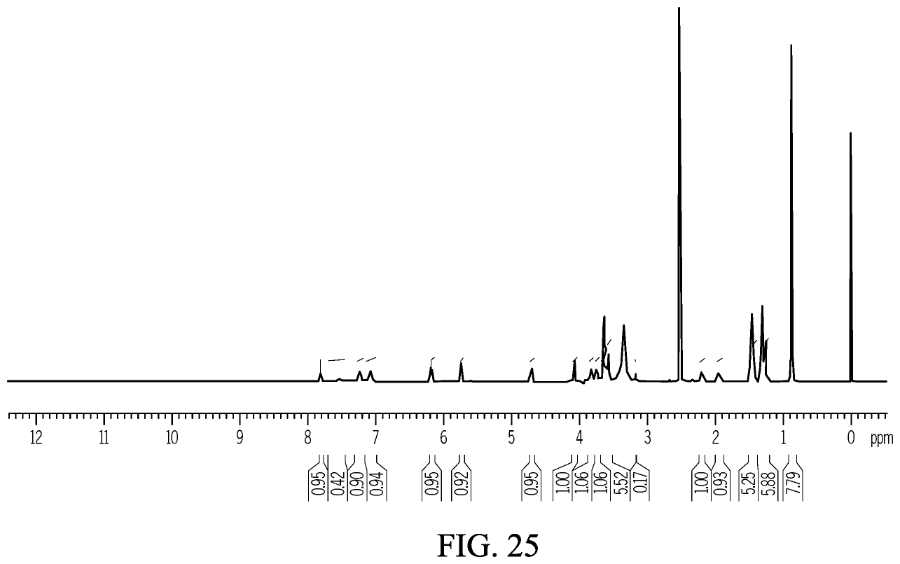

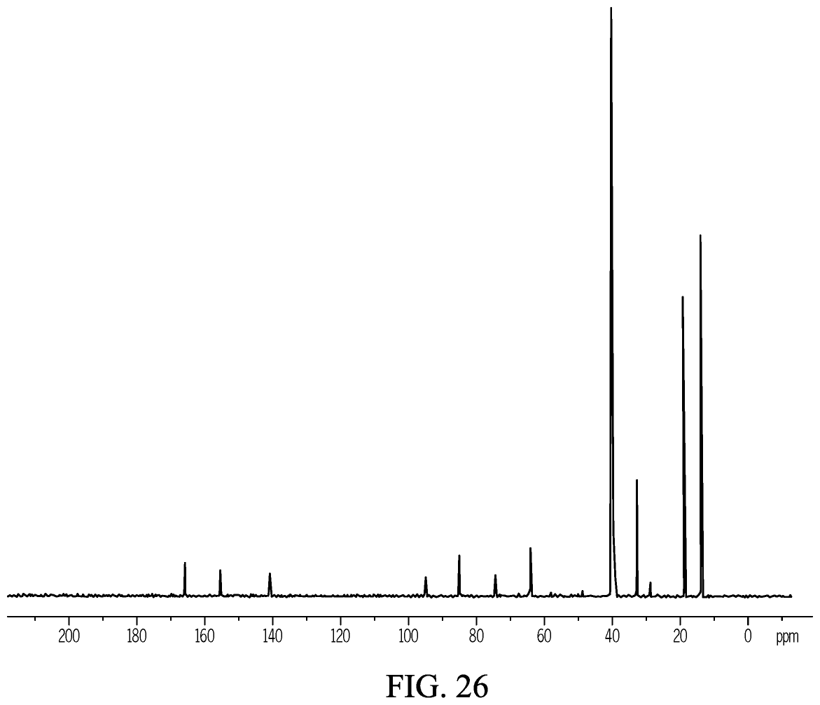

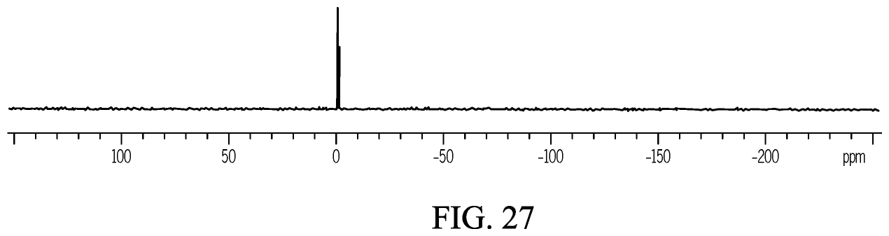

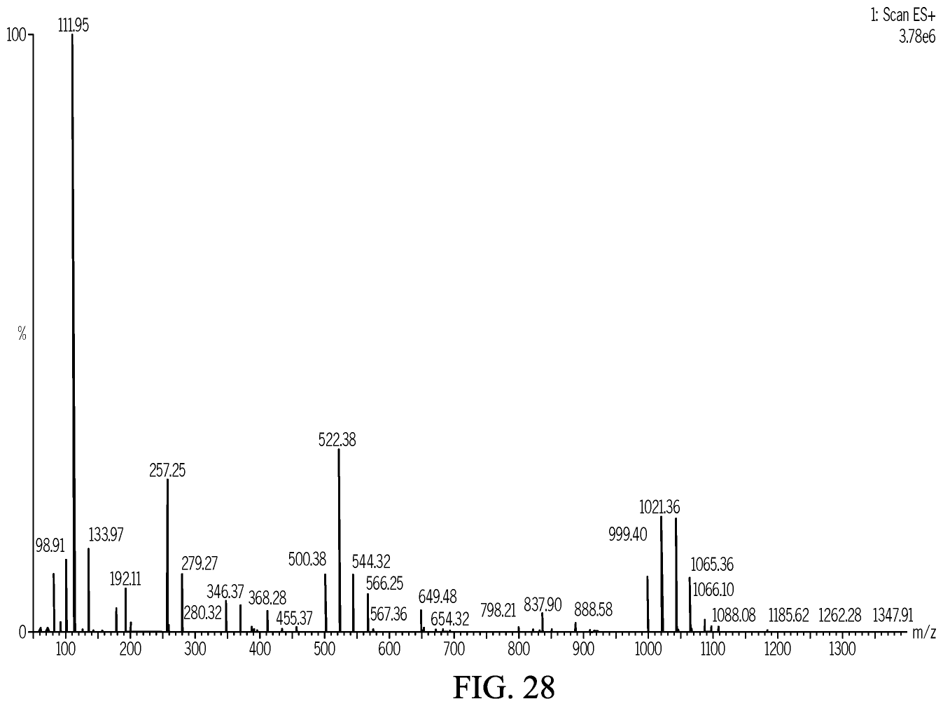

A flask was charged with a solution of 1.5 g (2.26 mmol, 1 equivalent) of 5-(4-(amino-2-oxopyridin-1(2H)-yl)-3-(butoxy(2,2,2-trifluoroethylprop-2-enyloxy)phosphoryloxy)tetra-hydrofuran-2-yl)methyl butyl-2,2,2-trifluoroethyl phosphate 49 in 7.5 ml of water and a solution of 0.78 g (4.5 mmol, 2.5 equivalents) of barium hydroxide was added. The reaction mixture was heated to 55° C. and stirred for 20 hours. The reaction mixture contained 41% of the product 14. FIG. 25 provides the ¹H NMR spectrum of compound 14 prepared by this method. FIG. 26 provides the ¹³C NMR spectrum of compound 14 prepared by this method. FIG. 27 provides the ³¹P NMR spectrum of compound 14 prepared by this method. FIG. 28 provides the mass spectrum of compound 14 prepared by this method. IR spectrum peaks: 3411, 2961, 1655, 1528, 1497, 1293, 1219, 1091, 1069, 1031, 976, 897, 826, 729, 562 cm⁻¹.

### Example 17

### 2,2,2-Trifluoroethy Butyl Iprop-2-Enyl Phosphate

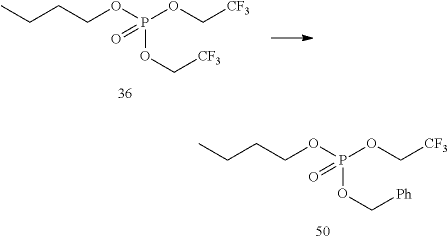 [SMILES: CCCCOP(=O)(OCC(F)(F)F)OCC(F)(F)F ; CCCCOP(=O)(OCc1ccccc1)OCC(F)(F)F]

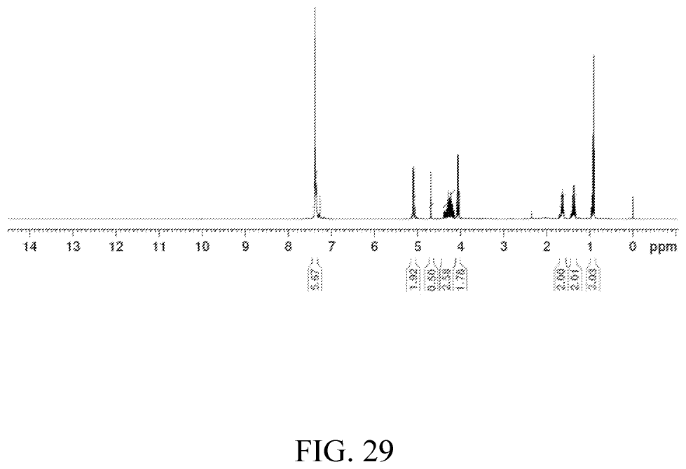

A flask was charged with 2 g (6.3 mmol, 1 equivalent) of bis-trifluoroethyl butyl phosphate 36 in 30 ml of toluene and cooled to 0° C. To the cooled solution was added 0.96 g (6.3 mmol, 1 equivalent) of 1,8-diazabicyclo[5.4.0]undec-7-ene and 0.7 g (6.3 mmol, 1 equivalent) of benzyl alcohol. The reaction mixture was allowed to come to room temperature overnight. The mixture was quenched by the addition of phosphate buffer (pH 7) at 0° C. The mixture was extracted twice with methyl t-butyl ether. The combined extracts were washed with aqueous brine, dried over sodium sulphate, and concentrated in vacuo to yield 33 1 g (48% yield) of 2,2,2-trifluoroethyl butyl benzyl phosphate 50. FIG. 29 provides the ¹H NMR spectrum of compound 50. LCMS data: Retention Time (RT) 3 min, 42.8%, M+1=327 (target product 50).

### Example 18

### Preparation of 5-(4-[(Phenylmethoxy)Carbonyl]-Amino-2-Oxopyridin-1(2H)-Yl)-3-(Butoxy(2,2,2-Trifluoroethyl-Benzyloxy)Phosphoryloxy)Tetrahydrofuran-2-Yl)Methyl Butyl-2,2,2-Trifluoroethyl Phosphate

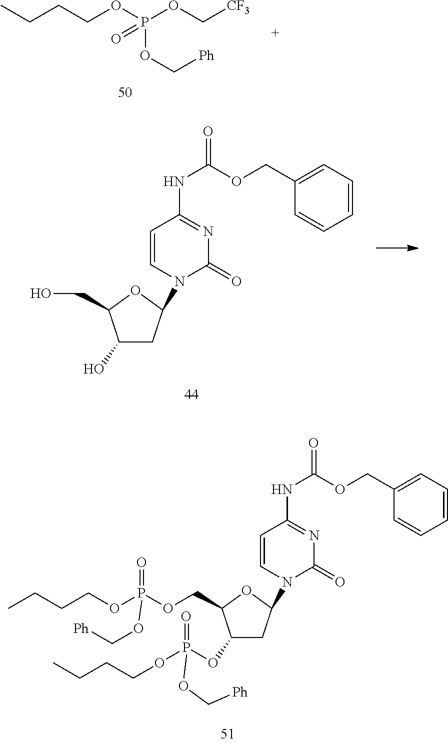 [SMILES: CCCCOP(=O)(OCc1ccccc1)OCC(F)(F)F ; O=C(Nc1ccn([C@H]2C[C@H](O)[C@@H](CO)O2)c(=O)n1)OCc1ccccc1 ; CCCCOP(=O)(OCc1ccccc1)OC[C@H]1O[C@@H](n2ccc(NC(=O)OCc3ccccc3)nc2=O)C[C@@H]1OP(=O)(OCCCC)OCc1ccccc1]

A flask was charged with 0.5 g (1.4 mmol, 1 equivalent) of 2′-deoxy-N-[(phenylmethoxy)carbonyl]-cytidine phosphate 44 in 7.5 ml of THF and cooled to 0° C. To the cooled solution was added 1 g (5.6 mmol, 4 equivalents) of sodium hexamethyldisilazide and 2.75 g (8.4 mmol, 6 equivalents) of 2,2,2-trifluoroethyl butyl benzyl phosphate 50. The reaction mixture was allowed to come to room temperature overnight. The mixture was quenched by the addition of phosphate buffer (pH 7) at 0° C. The mixture was extracted twice with methyl t-butyl ether. The combined extracts were washed with aqueous brine, dried over sodium sulphate, and concentrated in vacuo to yield 1 g of crude material containing 29% of 5-(4-[(phenylmethoxy)carbonyl]-amino-2-oxopyridin-1(2H)-yl)-3-(butoxy(2,2,2-trifluoroethyl-benzyloxy)phosphoryloxy)tetrahydrofuran-2-yl)methyl butyl-2,2,2-trifluoroethyl phosphate 51. LCMS data: Retention Time (RT) 6.3 min, M+1=815 (target product 51).

### Example 19

### Preparation Of 2′-Deoxy-N-[Bis(Tertiary Butyloxycarbonyl)]-Cytidine

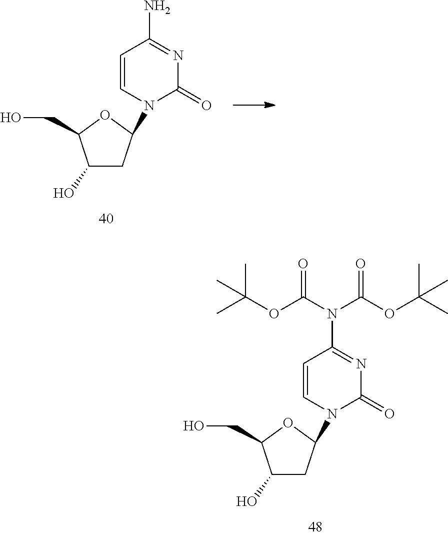 [SMILES: Nc1ccn([C@H]2C[C@H](O)[C@@H](CO)O2)c(=O)n1 ; CC(C)(C)OC(=O)N(C(=O)OC(C)(C)C)c1ccn([C@H]2C[C@H](O)[C@@H](CO)O2)c(=O)n1]

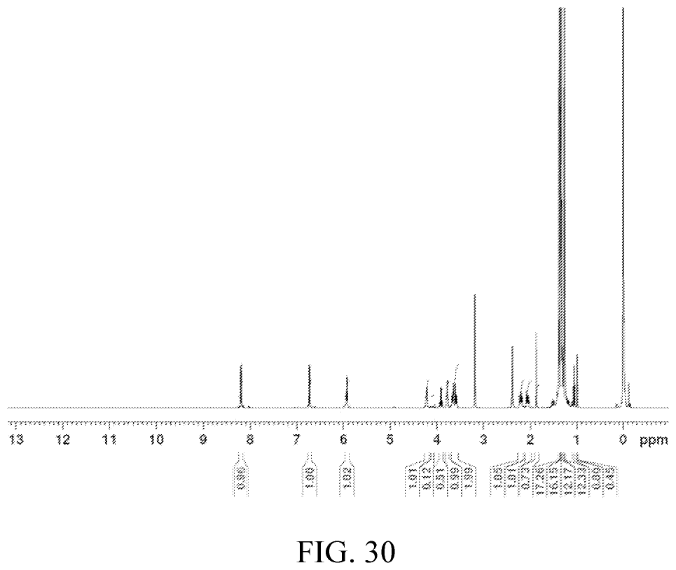

A flask was charged with 150 ml of acetonitrile and 50 g (0.22 mol, 1 equivalent) of 2′-deoxycytidine 40 and 106.5 g (0.66 mol, 3 equivalents) of hexamethyldisilazane. The solution was cooled to 10° C. and 5.4 g (0.044 mol, 0.2 equivalents) of dimethylaminopyridine and 1.46 g (0.007 mol, 0.3 equivalents) of trimethylsilyl triflate was added. The homogeneous solution was heated to 40° C. for 2 hours and then cooled to 20° C. An addition funnel was charged with 384 g (1.76 mol, 8 equivalents) of tertiarybutylcarbonyl anhydride, and the reagent was added over 3-4 hours maintaining the temperature at 20° C. The solution was stirred for an additional 6 hours monitoring the temperature to keep it below 30° C. Once the reaction was complete 450 ml of methanol was added and the solution was cooled to 10° C. An addition funnel was charged with 225 ml of triethylamine and the reagent was added over 2-3 hours maintaining the temperature at 20-25° C. Once the conversion was complete the solvents were removed under vacuum and the product was slurried in MTBE, filtered, and dried to give 67 g (71% yield) of 2′-deoxy-N-[bis(tertiary butyloxycarbonyl)]-cytidine 48. FIG. 30 provides the ¹H NMR spectrum of compound 48. LCMS data: Retention Time (RT) 2.7 min, 95.9%, M+1=428 (target product 48).

### Example 20

### Preparation of n-Butyl Tertiary Butyl 2,2,2-Trifluoroethyl Phosphate

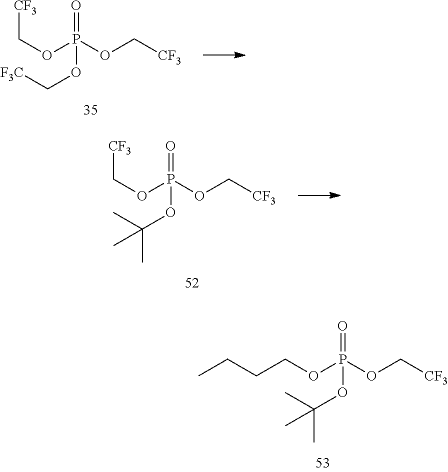 [SMILES: O=P(OCC(F)(F)F)(OCC(F)(F)F)OCC(F)(F)F ; CC(C)(C)OP(=O)(OCC(F)(F)F)OCC(F)(F)F ; CCCCOP(=O)(OCC(F)(F)F)OC(C)(C)C]

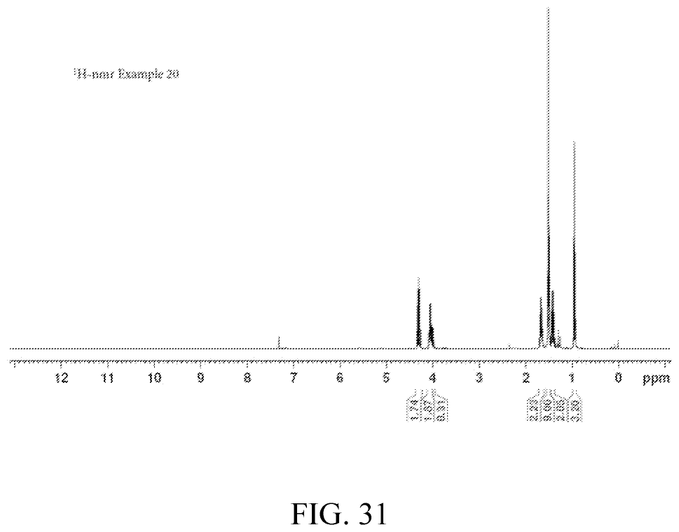

A flask was charged with 30 g (87 mmol, 1 equivalent) of bis-trifluoroethyl butyl phosphate 35 in 600 ml of toluene and cooled to −45° C. To the cooled solution was added 9.77 g (122 mmol, 1.4 equivalents) of 1M lithium tertiary butoxide in THF. The reaction mixture was allowed to come to room temperature overnight. Without isolating intermediate 52, an additional 7.7 g (94.6 mmol, 1.1 equivalents) of 1M lithium tertiary butoxide in THF was added and the mixture heated to 45° C. After 2 hours at 45° C., the mixture was quenched by the addition of phosphate buffer (pH 7) at 0° C. The mixture was extracted twice with methyl t-butyl ether. The combined extracts were washed with aqueous brine, dried over sodium sulphate, and concentrated in vacuo to yield 17 g (92% yield) of n-butyl t-butyl 2,2,2-trifluoroethyl phosphate 53. FIG. 31 provides the ¹H NMR spectrum of compound 53.

### Example 21

### Preparation of 5-(4-[(Phenylmethoxy)Carbonyl]-Amino-2-Oxopyridin-1(2H)-Yl)-3-(Butoxy(Hydoxy)Phosphoryloxy)Tetrahydrofuran-2-Yl)Methyl Butyl Phosphonate Bis Triethylamine Salt

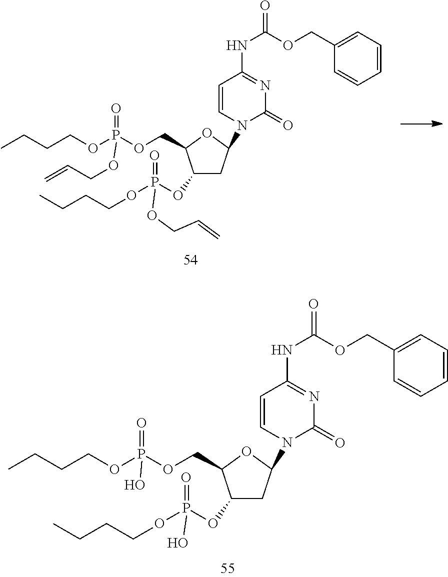 [SMILES: C=CCOP(=O)(OCCCC)OC[C@H]1O[C@@H](n2ccc(NC(=O)OCc3ccccc3)nc2=O)C[C@@H]1OP(=O)(OCC=C)OCCCC ; CCCCOP(=O)(O)OC[C@H]1O[C@@H](n2ccc(NC(=O)OCc3ccccc3)nc2=O)C[C@@H]1OP(=O)(O)OCCCC]

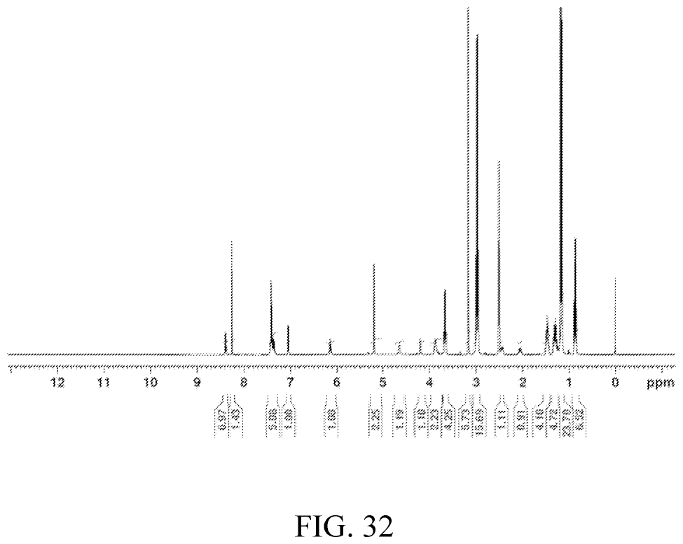

A flask was charged with a solution of 9 g (12.6 mmol, 1 equivalent) 54 in 135 ml of THF and 0.15 g (0.13 mmol, 100 equivalent) of tetrakis(triphenylphosphine)palladium was added. To the stirred solution was added 3.2 g (20.7 mmol, 1.66 equivalents and 3 g (69 mmol, 5.5 equivalents) of formic acid and the solution heated at 45-50° C. for 3 h. The solution was diluted with 10 ml water and washed with 20 ml of dichloromethane. The aqueous layer was lyophilized to yield 10 g (95% yield) of the product 55 as the bis triethylamine salt. FIG. 32 provides the ¹H NMR spectrum of compound 55 prepared according to this method. LCMS data: Retention Time (RT) 1.86 min, 97.2%, M+1=634 (target product 55).

While embodiments have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

1. A method for synthesizing a Bisphosphocin of Formula 1 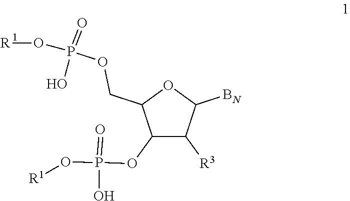
or a Bisphosphocin of Formula 2 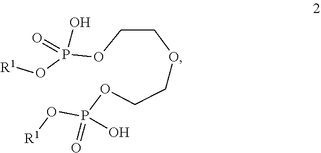
the method comprising:
contacting tris(trifluoroethyl)phosphate 3 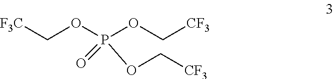 [SMILES: O=P(OCC(F)(F)F)(OCC(F)(F)F)OCC(F)(F)F]
with an alkyl alcohol R¹—OH under conditions sufficient to form a first mixed phosphate ester 4 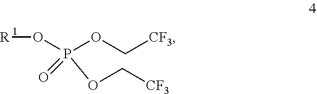
thereby producing the first mixed phosphate ester 4,
contacting the first mixed phosphate ester 4 with a lithium alkoxide LiOR² or an allyl alcohol HOR² under conditions sufficient to form a second mixed phosphate ester 5 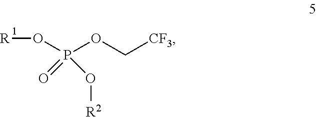
thereby producing the second mixed phosphate ester 5;
contacting the second mixed phosphate ester 5 with a dialcohol of Formula 6 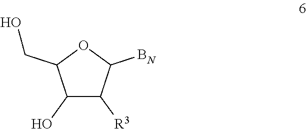
or of Formula 7 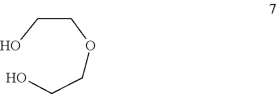
under conditions sufficient to form a protected Bisphosphocin of Formula 8 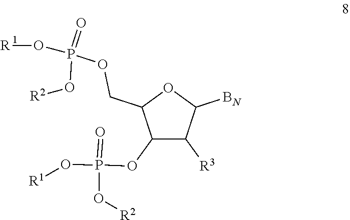
or of Formula 9 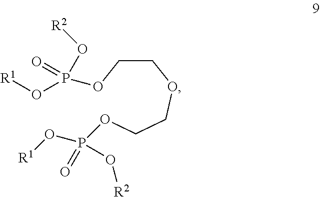
respectively, thereby producing the protected Bisphosphocin of Formula 8 or of Formula 9, respectively; and
deprotecting the protected Bisphosphocin of Formula 8 or of Formula 9 under conditions sufficient to form the Bisphosphocin of Formula 1 or of Formula 2, respectively, thereby producing the Bisphosphocin of Formula 1 or of Formula 2, respectively;
wherein:
each R¹ is independently (CH₂)ₙCH₃ or (CH₂)ₙOH;
each n is independently 2, 3, 4, 5, 6, 7, or 8;
each R² is independently (CH₃)₃C—, CF₃CH₂—, PhCH₂—, CH₂═CHCH₂—, (CH3)₂CH—, CCl₃CH₂—, (CH3)₃SiCH₂CH₂—, 4-methoxy benzyl, C₆H₅SCH₂CH₂—, CH₃SO₂CH₂CH₂—, CH₃SCH₂CH₂CH₂CH₂—, and CF₃C(═O)N(CH₃)CH₂CH₂CH₂CH₂—;
each R³ is independently hydrogen or methoxy; and
B_{N} is a nitrogenous base.

2. The method of claim 1, wherein the nitrogenous base comprises a purine, a pyrimidine, or a derivative thereof.

3. The method of claim 1, wherein the nitrogenous base is selected from the group consisting of adenine, cytosine, guanine, thymine, and uracil.

4. The method of claim 1, wherein the Bisphosphocin of Formula 1 or the Bisphosphocin of Formula 2 is selected from the group consisting of a compound of Formula 10 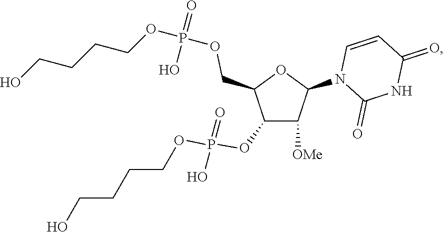 [SMILES: CO[C@@H]1[C@H](OP(=O)(O)OCCCCO)[C@@H](COP(=O)(O)OCCCCO)O[C@H]1n1ccc(=O)[nH]c1=O]
a compound of Formula 11 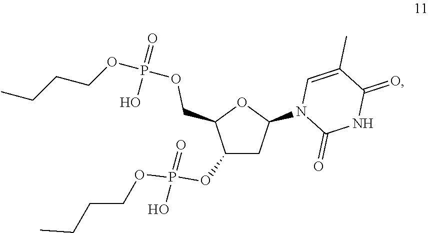 [SMILES: CCCCOP(=O)(O)OC[C@H]1O[C@@H](n2cc(C)c(=O)[nH]c2=O)C[C@@H]1OP(=O)(O)OCCCC]
a compound of Formula 12 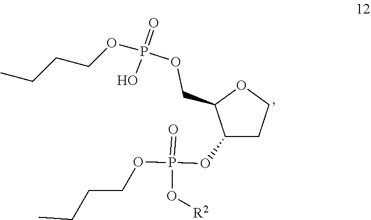
a compound of Formula 13 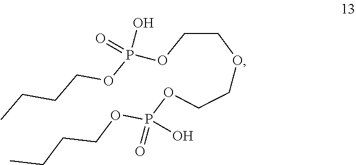 [SMILES: CCCCOP(=O)(O)OCCOCCOP(=O)(O)OCCCC]
and a compound of Formula 14

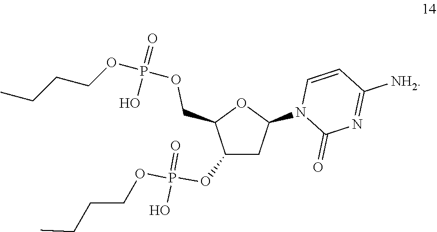 [SMILES: CCCCOP(=O)(O)OC[C@H]1O[C@@H](n2ccc(N)nc2=O)C[C@@H]1OP(=O)(O)OCCCC]

5. The method of claim 1, wherein contacting tris(trifluoroethyl)phosphate 3 with the alkyl alcohol R¹—OH comprises:
dissolving the tris(trifluoroethyl)phosphate 3 in a solvent to form a first solution;
adding a non-nucleophilic base to the first solution;
adding the alkyl alcohol R¹—OH to the first solution; and
maintaining a temperature of the first solution from about −50° C. to about 50° C.

6. The method of claim 5, wherein contacting the first mixed phosphate ester 4 with the lithium alkoxide LiOR² or the allyl alcohol HOR² comprises:
dissolving the first mixed phosphate ester 4 in a solvent to form a second solution;
adding a non-nucleophilic base to the second solution;
adding the lithium alkoxide LiOR² or the allyl alcohol HOR² to the second solution; and
maintaining a temperature of the second solution from about −50° C. to about 50° C.

7. The method of claim 6, wherein contacting the second mixed phosphate ester 5 with a dialcohol of Formula 6 or of Formula 7 comprises:
dissolving the dialcohol of Formula 6 or of Formula 7 in a solvent to form a third solution;
adding an acid or a base to the third solution;
adding the second mixed phosphate ester 5 to the third solution; and
maintaining a temperature of the third solution from about −50° C. to about 50° C.

8. The method of claim 7, wherein deprotecting the protected Bisphosphocin of Formula 8 or of Formula 9 comprises:
dissolving the protected Bisphosphocin of Formula 8 or of Formula 9 in a solvent to form a fourth solution;
adding an deprotection agent to the fourth solution; and
maintaining a temperature of the fourth solution from about 40° C. to about 140° C.

9. The method of claim 1, wherein the dialcohol of Formula 6 is a dialcohol of Formula 17 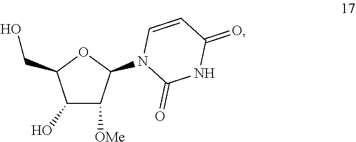 [SMILES: CO[C@@H]1[C@H](O)[C@@H](CO)O[C@H]1n1ccc(=O)[nH]c1=O]
and the Bisphosphocin of Formula 1 is a Bisphosphocin of Formula 10

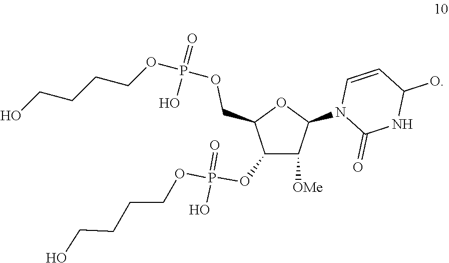

10. The method of claim 1, wherein the dialcohol of Formula 6 is a dialcohol of Formula 18 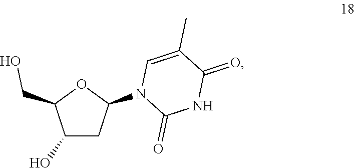 [SMILES: Cc1cn([C@H]2C[C@H](O)[C@@H](CO)O2)c(=O)[nH]c1=O]
and the Bisphosphocin of Formula 1 is a Bisphosphocin of Formula 11

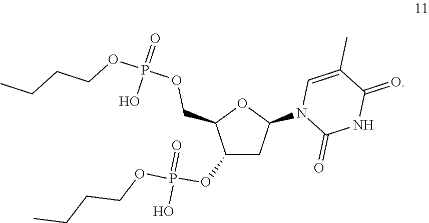 [SMILES: CCCCOP(=O)(O)OC[C@H]1O[C@@H](n2cc(C)c(=O)[nH]c2=O)C[C@@H]1OP(=O)(O)OCCCC]

11. The method claim 1, wherein the dialcohol of Formula 6 is a dialcohol of Formula 19 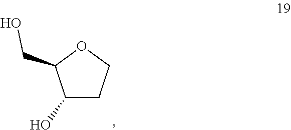
and the Bisphosphocin of Formula 1 is a Bisphosphocin of Formula 12

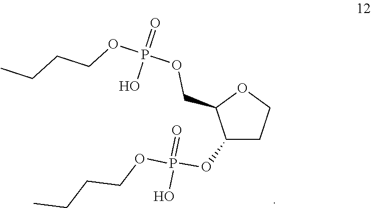 [SMILES: CCCCOP(=O)(O)OC[C@H]1OCC[C@@H]1OP(=O)(O)OCCCC]

12. The method of claim 1, wherein the dialcohol is a dialcohol of Formula 7 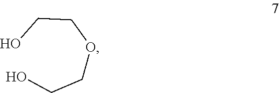
and the Bisphosphocin of Formula 2 is a Bisphosphocin of Formula 13

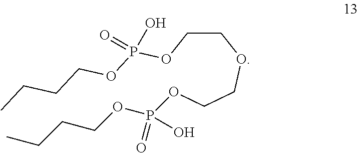 [SMILES: CCCCOP(=O)(O)OCCOCCOP(=O)(O)OCCCC]

13. The method of claim 1, wherein the dialcohol of Formula 6 is a dialcohol of Formula 22 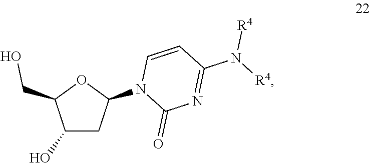
and the Bisphosphocin of Formula 1 is a Bisphosphocin of Formula 14 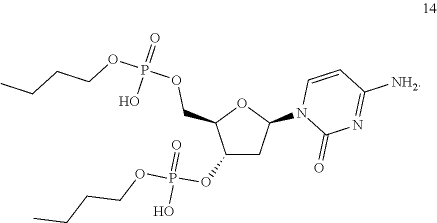
wherein each R⁴ is independently hydrogen, benzyloxycarbonyl, trichloroethoxycarbonyl, t-butoxycarbonyl, benzoyl, acetyl, and 9-fluorenylmethoxycarbonyl.

14. A method for synthesizing a Bisphosphocin of Formula 1 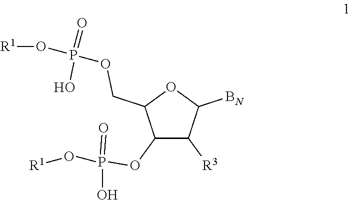
or a Bisphosphocin of Formula 2 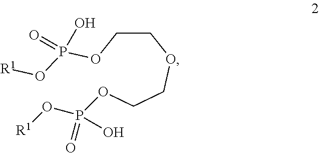
the method comprising:
contacting a dialcohol of Formula 6 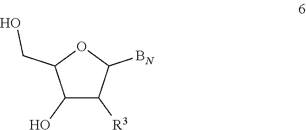
or of Formula 7 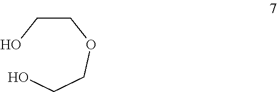
with phosphorus oxychloride in the presence of an alcohol of formula HO(CH₂)ₙCH₃ or HO(CH₂)ₙOH, under conditions sufficient to form the Bisphosphocin of Formula 1 or a Bisphosphocin of Formula 2, respectively;
wherein
each R¹ is independently (CH₂)ₙCH₃ or (CH₂)ₙOH;
each n is independently 2, 3, 4, 5, 6, 7, or 8;
each R³ is independently hydrogen or methoxy; and
B_{N} is a nitrogenous base.

15. The method of claim 14, wherein the nitrogenous base comprises a purine or a pyrimidine.

16. The method of claim 14, wherein the nitrogenous base is selected from the group consisting of adenine, cytosine, guanine, thymine, and uracil.

17. The method of claim 14, wherein the Bisphosphocin of Formula 1 or the Bisphosphocin of Formula 2 is selected from the group consisting of a compound of Formula 10 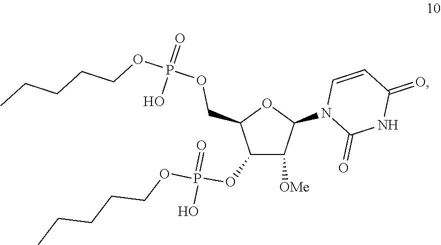 [SMILES: CCCCCOP(=O)(O)OC[C@H]1O[C@@H](n2ccc(=O)[nH]c2=O)[C@H](OC)[C@@H]1OP(=O)(O)OCCCCC]
a compound of Formula 11 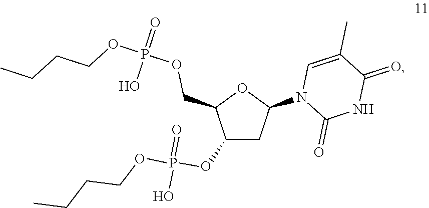 [SMILES: CCCCOP(=O)(O)OC[C@H]1O[C@@H](n2cc(C)c(=O)[nH]c2=O)C[C@@H]1OP(=O)(O)OCCCC]
a compound of Formula 12 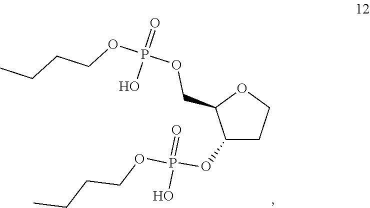 [SMILES: CCCCOP(=O)(O)OC[C@H]1OCC[C@@H]1OP(=O)(O)OCCCC]
adding the alcohol of formula HO(CH₂)ₙCH₃ or HO(CH₂)ₙOH to the mixture; and
stirring the mixture at a temperature from about −20° C. to about 20° C. for a period of time from 1 hour to 10 hours.

19. The method of claim 14, wherein the alcohol of formula HO(CH₂)ₙCH₃ is butanol.

20. The method of claim 14, wherein the alcohol of formula HO(CH₂)ₙOH is 1,4-butanediol.

21. The method of claim 14, wherein the dialcohol of Formula 6 is a dialcohol of Formula 17 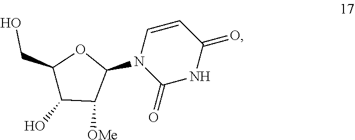 [SMILES: CO[C@@H]1[C@H](O)[C@@H](CO)O[C@H]1n1ccc(=O)[nH]c1=O]
and the Bisphosphocin of Formula 1 is a Bisphosphocin of Formula 10

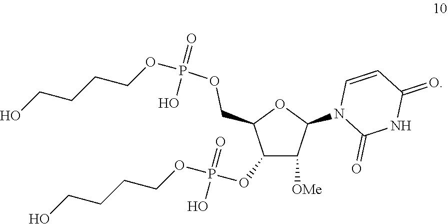 [SMILES: CO[C@@H]1[C@H](OP(=O)(O)OCCCCO)[C@@H](COP(=O)(O)OCCCCO)O[C@H]1n1ccc(=O)[nH]c1=O]

22. The method of claim 14, wherein the dialcohol of Formula 6 is a dialcohol of Formula 18
a compound of Formula 13 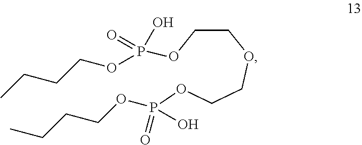 [SMILES: CCCCOP(=O)(O)OCCOCCOP(=O)(O)OCCCC]
a compound of Formula 14 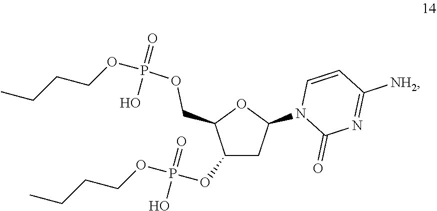 [SMILES: CCCCOP(=O)(O)OC[C@H]1O[C@@H](n2ccc(N)nc2=O)C[C@@H]1OP(=O)(O)OCCCC]
and a compound of Formula 23:

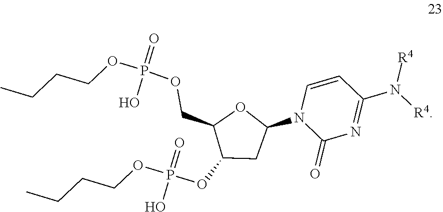

18. The method of claim 1, wherein contacting the dialcohol of Formula 6 or of Formula 7 with the phosphorus oxychloride comprises:
dissolving the dialcohol of Formula 6 or of Formula 7 in a mixture of trialkyl phosphate and phosphorus oxychloride;
stirring the mixture at a temperature from about −20° C. to about 20° C. for a period of time from about 10 minutes to about 3 hours; 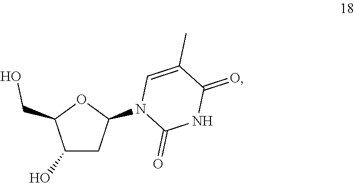 [SMILES: Cc1cn([C@H]2C[C@H](O)[C@@H](CO)O2)c(=O)[nH]c1=O]
and the Bisphosphocin of Formula 1 is a Bisphosphocin of Formula 11

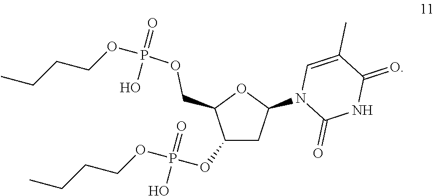 [SMILES: CCCCOP(=O)(O)OC[C@H]1O[C@@H](n2cc(C)c(=O)[nH]c2=O)C[C@@H]1OP(=O)(O)OCCCC]

23. The method of claim 14, wherein the dialcohol of Formula 6 is a dialcohol of Formula 19 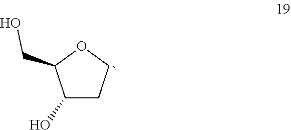
and the Bisphosphocin of Formula 1 is a Bisphosphocin of Formula 12

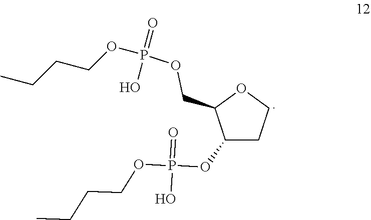 [SMILES: CCCCOP(=O)(O)OC[C@H]1OCC[C@@H]1OP(=O)(O)OCCCC]

24. The method of claim 14, wherein the dialcohol is a dialcohol of Formula 7 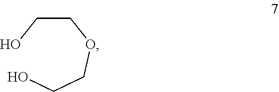
and the Bisphosphocin of Formula 2 is a Bisphosphocin of Formula 13

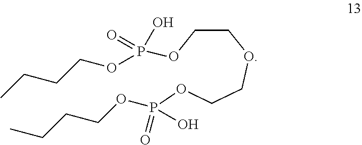 [SMILES: CCCCOP(=O)(O)OCCOCCOP(=O)(O)OCCCC]

25. The method of claim 14, wherein the dialcohol of Formula 6 is a dialcohol of Formula 22 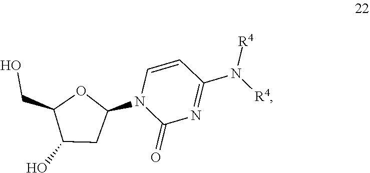
and the Bisphosphocin of Formula 1 is a Bisphosphocin of Formula 23 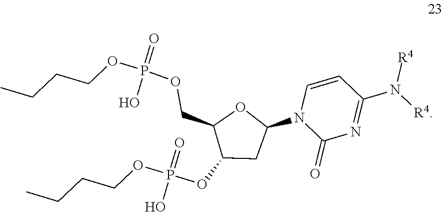
wherein each R⁴ is independently hydrogen, benzyloxycarbonyl, trichloroethoxycarbonyl, t-butoxycarbonyl, benzoyl, acetyl, and 9-fluorenylmethoxycarbonyl.

26. The method of claim 25, further comprising deprotecting the Bisphosphocin of Formula 23 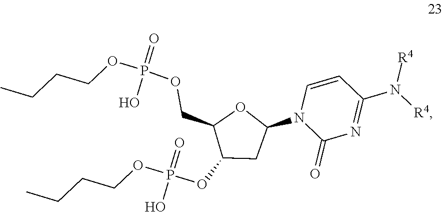
thereby producing a Bisphosphocin of Formula 14: 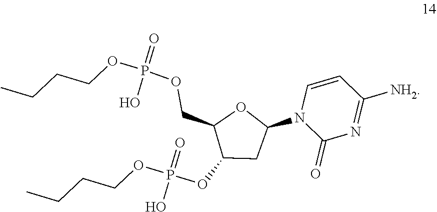 [SMILES: CCCCOP(=O)(O)OC[C@H]1O[C@@H](n2ccc(N)nc2=O)C[C@@H]1OP(=O)(O)OCCCC]
